(19) United States
(12) Patent Application Publication
(10) Pub. No.: US 2005/0148036 A1
(43) Pub. Date: Jul. 7, 2005

(54) Method for detecting a mononuclear cell phenotype related to a pro-tumor immune response

(75) Inventors: Emilio Barbera-Guillem, Powell, OH (US); M. Bud Nelson, Worthington, OH (US)

Correspondence Address: BENESCH, FRIEDLANDER, COPLAN & ARONOFF LLP;ATTN: IP DEPARTMENT DOCKET CLERK, 2300 BP TOWER, 200 PUBLIC SQUARE, CLEVELAND, OH, 44114, US

(73) Assignee: BioCrystal Ltd., Westerville, OH (US)

(21) Appl. No.: 10/997,648

(22) Filed: Nov. 24, 2004

Related U.S. Application Data
(63) Continuation of application No. 09/792,561, filed on Feb. 23, 2001.
Application No. 09/792,561 is a continuation-in-part of application No. 09/435,289, filed on Nov. 5, 1999, now Pat. No. 6,251,616.
Application No. 09/435,289 is a continuation-in-part of application No. 09/333,103, filed on Jun. 15, 1999, now abandoned.
(60) Provisional application No. 60/115,946, filed on Jan. 14, 1999.
(60) Provisional application No. 60/117,895, filed on Jan. 29, 1999.

(51) Int. Cl. C12N 5/00; G01N 33/574

(52) U.S. Cl. 435/7.23

(57) ABSTRACT

A method for screening an individual for a pathological condition comprising a pro-tumor immune response by assaying a clinical sample, obtained from the individual, with a plurality of affinity ligands for detecting and determining an amount of mononuclear cell phenotype. The amount of mononuclear cell phenotype determined in the clinical sample is then compared to a reference value for the mononuclear cell phenotype, wherein a difference in the amount of mononuclear cell phenotype determined from the clinical sample as compared to the reference value comprises an indicator of the presence of a pro-tumor immune response.

This is a continuation-in-part based on application Ser. No. 09/435,289, which is a continuation-in-part application based on application Ser. No. 09/333,103 which is based on provisional Application Nos. 60/115,946 and 60/117,895; all of which are herein substantially incorporated by reference.

### FIELD OF THE INVENTION

The present invention is related to biological testing, and in particular to methods for determining the amount of mononuclear cell phenotype comprising one or more selected subpopulations of mononuclear cells which may be associated with, or an indicator for, the progression of solid, non-lymphoid tumors. A mononuclear cell phenotype comprises an amount of one or more lymphocyte subpopulations, one or more follicular dendritic cell subpopulations, and a combination thereof; each subpopulation of which may be used singly, or in combination with other subpopulations, as a diagnostic indicator in a method for screening for the presence of a pro-tumor immune response in humans, and as a prognostic indicator in a method for screening for the potential of tumor development or recurrence.

### BACKGROUND OF THE INVENTION

1. B Cells

The response of an individual to tumor cells involves the reactions and counteractions mediated by both cellular and humoral arms of the immune system. Tumor cell growth may represent a disturbance in the equilibrium of the immune system that is pre-existing, and/or induced by the tumor cells themselves. However, most investigations to date have focused on the role of T cells in tumor immunity. The role of B cells in a tumor-bearing individual still remains unclear.

Previous studies have shown that lymph nodes regional to a primary tumor in cancer patients, and in in vivo experimental animal models of tumor development, can undergo a prominent expansion in the germinal centers. A recent study confirmed that there is an increase in the number of B cells in the germinal centers of lymph nodes regional to primary tumors. The number of B cells increase in the regional lymph nodes concomitantly with tumor development, and it has been reported that B cells appear to be able to elicit anti-tumor immunity by producing antitumor antibodies (see, e.g., Carey et al., 1976, Proc. Natl. Acad. Sci. USA 73: 3278-3282; Abe et al., 1989, Cancer Res. 80: 271-276; Christensen et al., 1989, Int. J. Cancer 37: 683-688).

However, unlike the pattern found in the lymph nodes, the percentage of B lymphocyte populations in the blood of cancer patients are similar to the values found in healthy controls (Eremin et al., 1976, Int. Archs Allergy appl. Immun. 52: 277-290; Svennevig et al., 1979, Int. J. Cancer 23: 626-631). Some studies report a lower mean percentage of circulating B lymphocytes in cancer patients as compared to the mean percentage in apparently normal individuals (Wood and Neff, 1978, J. Natl. Cancer Inst. 61: 715-718). In these latter studies, the low values of circulating B lymphocytes were observed both in the absence of therapy, and in the presence of chemotherapy or radiation therapy; and further, could not be found to correlate with the stage of disease. More recently, the percentage of a specific subpopulation of B lymphocytes, identified as CD5+ and also known as B1 cells, appears to be slightly increased in the peripheral blood of cancer patients as compared to the values in healthy controls (Stein et al., 1991, Clin. Exp. Immunol. 85: 418-23). It is noted that CD5+ B cells are a different cell subset than memory B cells (CD5−; Brown, 1992, Crit. Rev. Immunol. 11: 395-417). While it appears that a humoral immune response towards tumor-associated antigens can be mounted in cancer patients, the role of the B cells in the host response to tumor, and any significance relative to the detection of B cells in the host response to tumor, remain poorly defined.

2. T Cells

T cell subsets, mainly CD4+ cells and CD8+ cells, have been studied in individuals having solid, nonlymphoid tumor. In general, regional lymph nodes close to (e.g., draining) a primary solid, nonlymphoid tumor, and the nodes involved with metastases thereof, show a significant decrease of CD4+ T cells (see, e.g., Takemura et al., 1991, Cancer J. 4: 244-248). As to peripheral blood values, it is a general observation that activated CD4+ T cells (CD4+, HLA DR+) may be significantly higher in amounts in Stage I patients than that observed in healthy controls, but that the CD4+ subset becomes significantly decreased during advanced stages of malignancy. In patients with bladder cancer, the absolute number of CD11b+CD8+ cells (suppressor T lymphocytes) in peripheral blood correlated inversely with histological grade. Additionally, there was a significantly lower absolute number of peripheral blood CD11b−CD8+ cells (cytotoxic T lymphocytes) in patients with invasive bladder cancer as compared to that in patients with superficial bladder cancer (see, e.g., Ono et al., 1996, Reg. Cancer Treat. 9: 40-43). It has also been reported that radiation therapy for primary cancer results in reduced B lymphocytes and reduced T lymphocytes in proportion to their total lymphocyte population, so that their percentages remain unchanged.

3. Follicular Dendritic Cells

Dendritic cells are a population of antigen presenting cells that comprise multiple distinct subpopulations. Of the distinct subpopulations, follicular dendritic cells (DRC-1, KIM4+; “FDC”) reside in germinal centers within lymphoid follicles of secondary lymphoid tissues. FDC have a distinctive ability to trap and retain unprocessed antigen, in the form of immune complexes, in a spacial arrangement for effective antigen presentation to B cells. Hence, FDC are the main antigen presenting cells to B cells in the germinal center, and play a major role in inducing B cell proliferation in lymph nodes. Precursors of FDC may be present in low numbers in blood and bone marrow (Haley et al., 1995, Adv. Exp. Med. Biol. 378: 289-91). For example, in the non-adherent mononuclear blood cell fraction, separated at a density of 1.077 g/ml in a density gradient, only 0.1 per million of the cells revealed staining with dendritic cell marker Ki-M4 (Parwaresch et al., 1983, Blood 62: 585-90).

We have discovered that certain soluble tumor antigens, shed from tumor cells of solid, non-lymphoid tumors, are capable of inducing an immune response which promotes tumor progression (one or more of tumor growth, invasion, and metastasis). Additionally, we have developed various compositions and methods for treating a pro-tumor immune response.

Therefore, a need exists for methods which may be used to screen for the possible presence of a pro-tumor immune response in an individual; particularly in an individual who has a solid, non-lymphoid tumor, or an individual who is at high risk (e.g., environmentally and/or genetically) for developing a solid, non-lymphoid tumor, or an individual who has been treated for a solid, non-lymphoid tumor and thereby inherently carries a risk of recurrence.

### SUMMARY OF THE INVENTION

According to a primary object of the present invention, determined is an amount of mononuclear cell phenotype by a method in which one or more determinants, expressed by cells of the one or more selected subpopulations comprising mononuclear cell phenotype, is specifically bound by one or more affinity ligands, thereby facilitating determination of an amount of mononuclear cell phenotype.

It is another object of the present invention to provide a method for screening for a pro-tumor immune response in an individual by determining an amount of mononuclear cell phenotype present in a sample of lymphoid tissue regional or distal to an organ which is (or was, in the case of resection) the site of primary tumor in the individual. The amount of a mononuclear cell phenotype determined from the sample may comprise an indicator for the presence of a pro-tumor immune response; and hence may be of diagnostic value.

It is an additional object of the present invention to provide a method for screening for a pro-tumor immune response in an individual by determining an amount of mononuclear cell phenotype in one or more samples of body fluid of the individual (e.g., peripheral blood, or an effusion). The amount a mononuclear cell phenotype determined from the sample may comprise an indicator for the presence of a pro-tumor immune response; and hence may be of diagnostic value.

It is another object of the present invention to provide a method for providing a prognosis for an individual having a pro-tumor immune response by determining an amount of mononuclear cell phenotype present in a clinical sample obtained from the individual (“test sample”), and comparing the amount of mononuclear cell phenotype with a reference value (predetermined normal control value, or earlier value from the same individual) wherein the a difference between the test value and the reference value may comprise an indicator for the state of the pro-tumor immune response; and hence may be of prognostic value.

As described in U.S. Pat. No. 6,194,213, assay kits may be used for performing the above described methods. The assay kits may include various components, depending on the complexity of the type of method utilized for determining an amount of mononuclear cell phenotype. Briefly, an assay kit would typically contain one or more reagents, with each reagent comprising an affinity ligand capable of binding to a determinant that is expressed by mononuclear cell phenotype, and which facilitates detecting and quantitating (“determining”) mononuclear cell phenotype present in the sample analyzed. In a preferred embodiment, an assay kit may comprise components selected from the group consisting of one or more reagents for detecting B lymphocytes (e.g., by a pan B cell marker), one or more reagents for detecting T lymphocytes (e.g., by a pan T cell marker), one or more reagents for detecting a marker of a mononuclear cell subpopulation which is characteristic of a response to an immune process (a response may include, but is not limited to memory, activation, binding a shed tumor antigen (e.g., in antigen presentation), clonal expansion, mobilization, migration (e.g., in mediating adhesion or circulation), maturation (e.g., differentiation), and a combination thereof), one or more reagents for detecting FDC (e.g., by a pan FDC marker), and a combination thereof; and may further comprise a known amount of mononuclear cells for use in comparatively determining alterations in mononuclear cell phenotype in clinical samples (e.g., for use as one or more standards, one or more controls, or a combination thereof, in the method for determining an amount of mononuclear cell phenotype), instructions for use of the assay kit and components, and a combination thereof.

The foregoing objects are achieved because of: (a) the discovery of a novel mechanism, a pro-tumor immune response, that may be involved in the promotion of tumor progression; and (b) an unexpected demonstration that there may exist mononuclear cell phenotype (e.g., one or more subpopulations) that may be present and that may differ in amounts (“an alteration in mononuclear cell phenotype”) in individuals having a pro-tumor immune response, as compared to that present in individuals lacking a pro-tumor immune response (e.g., healthy individuals). Thus, determined from a clinical sample is an amount of mononuclear cell pheno-type, and the resultant amount may be used as in determining an indicator relative to a pro-tumor immune response which may be used for diagnostic or prognostic purposes. For example, a mononuclear cell phenotype comprising alterations in an amount of one or more B cell subpopulations determined from a clinical sample (the alteration comprising a difference in the amount determined from the sample as compared to a reference value for the one or more B cell subpopulations) may be an indicator for, and used as a screening tool for identifying, individuals that may have a pro-tumor immune response (e.g., tumor and a pro-tumor immune response, or a pro-tumor immune response in absence of detectable tumor). A mononuclear cell phenotype comprising alterations in an amount of one or more T cell subpopulations determined from a clinical sample may be an indicator for, and used as a screening tool for identifying, individuals that may have a pro-tumor immune response. A mononuclear cell phenotype comprising alterations in an amount of one or more follicular dendritic cell subpopulations determined from a clinical sample may be an indicator for, and used as a screening tool for identifying, individuals that may have a pro-tumor immune response. For purposes of diagnostic or prognostic use, the mononuclear cell phenotype may also comprise a combination of alterations (e.g., in amounts of one or more B cell subpopulations and of one or more T cell subpopulations; in amounts of one or more B cell subpopulations, of one or more T cell subpopulations, and of one or more FDC subpopulations; in amounts of one or more B cell subpopulations and of one or more FDC subpopulations; and in amounts of one or more T cell subpopulations and of one or more alterations in FDC subpopulations).

The above and other objects, features, and advantages of the present invention will be apparent in the following Detailed Description of the Invention when read in conjunction with the accompanying drawings.

### BRIEF DESCRIPTION OF THE DRAWINGS

FIG. 1 is a histogram illustrating CD21+ expression (Normal) as compared to CD21+ hyperexpression, termed “CD21++”, (Cancer) in B cells.

FIG. 2 is a bar graph illustrating an amount of sTn+(CD19−CD21+sTn+) FDC determined from lymphoid tissue of individuals having solid, non-lymphoid tumor and a pro-tumor immune response (“Cancer”) as compared to a reference value (amount in healthy controls; “Normal”).

FIG. 3 is a bar graph illustrating amounts of overall B cells (e.g., CD19+ cells), CD21 hyperexpressing memory B cells (e.g., CD19+CD21++ cells), and sTn+ B cells (e.g., CD19+sTn+ cells) after surgery but before chemotherapy (□) as compared to the respective amounts after chemotherapy (▪), and as compared with normal control values (“]N”)

FIG. 4 is a bar graph illustrating amounts of overall B cells (e.g., CD19+ cells), memory B cells (e.g., CD19+CD21+ cells), and sTn+ B cells (e.g., CD19+sTn+ cells) after anticancer treatment (▪), as compared with normal control values (“]N”).

FIG. 5 is a graph showing the depletion of overall B cells (e.g., CD19+ cells) effected by anticancer therapy.

FIG. 6 is a graph showing the depletion of sTn+ B cells (e.g., CD19+sTn+ cells) effected by anticancer therapy.

FIG. 7 is a graph showing the depletion of sTn+ memory B cells (e.g., CD19+CD21+sTn+ cells) effected by anticancer therapy.

FIG. 8 is a graph showing the depletion of memory B cells (e.g., CD19+CD21+ cells) effected by anticancer therapy.

### DETAILED DESCRIPTION OF THE INVENTION

Definitions

Note in describing embodiments of the present invention, such terms as “first” and “second” and the like are words of convenience in order to distinguish between different elements. Such terms as “first” and “second” are not intended to be limiting as to the sequence of a method or priority in which the different elements may be utilized.

The term “mononuclear cell phenotype” is used herein, for purposes of the specification and claims, to mean one or more mononuclear cell subpopulations comprising mononuclear cells which can be detected using a plurality of affinity ligands, wherein one or more first affinity ligands has binding specificity for a determinant selected from the group consisting of a pan B cell marker, a pan T cell marker, a pan follicular dendritic cell marker, and a combination thereof; and one or more second affinity ligands for detecting a mononuclear cell subpopulation expressing a determinant in response to an immune process (as known in the art, the immunological response may comprise memory, activation, binding a shed tumor antigen (e.g., in antigen presentation), clonal expansion, mobilization, migration (e.g., in mediating adhesion or circulation), maturation (e.g., differentiation), and a combination thereof; wherein such a determinant may include, but is not limited to, one or more of CD21, CD5, CD79 (a or b), CD75, CD45 (CD45Rahi or CD45RO), CD22, an epitope comprising a terminal 2,6-linked sialic acid (e.g., sTn), and the like); and in a preferred embodiment comprises sTn+ B cells, sTn+ B1 cells, memory B cells, sTn+ memory B cells, sTn+ T cells, sTn+ FDC, a combination thereof; and may also comprise mononuclear cells comprising a combination selected from the group consisting of an overall subpopulation of B cells (e.g., CD19+ cells) and one or more mononuclear cell subpopulations, an overall population of FDCs (e.g., CD19−CD21+ cells) and one or more mononuclear cell subpopulations, and a combination thereof.

The term “affinity ligand” is used herein, for purposes of the specification and claims, to mean a molecule which has binding specificity and avidity for a determinant associated with, and which can be used for diagnostic and/or prognostic detection of, mononuclear cell phenotype. For example, one type of affinity ligand, specific for a pan B cell marker (e.g., CD19+), may be used alone to detect an overall B cell subpopulation (e.g., CD19+ cells). This type of pan B cell marker may also be used in combination with other affinity ligands (e.g., specific for CD21+) to detect memory B cells (e.g., CD19+CD21+ cells). In general, affinity ligands are known to those skilled in the art to include, but are not limited to, lectins, antibodies, immunoreactive fragments produced or derivatives derived from antibodies, peptides, and aptamers (see, e.g., U.S. Pat. No. 5,789,157). Immunoreactive fragments produced or derivatives derived from an antibody molecule are fragments which retain all or a portion of the binding function of the whole antibody molecule, and are known to those skilled in the art to include F(ab′)₂, Fab′, Fab, Fv, scFV, Fd′ and Fd fragments. Methods for producing the various fragments from MAbs are well known in the art. For example, F(ab′)₂ can be produced by pepsin digestion of the monoclonal antibody, and Fab′ may be produced by reducing the disulfide bridges of F(ab′)₂ fragments. Fab fragments can be produced by papain digestion of the monoclonal antibody, whereas Fv can be prepared according to methods described in U.S. Pat. No. 4,642,334. Single chain antibodies can be produced as described in U.S. Pat. No. 4,946,778. In a preferred embodiment, affinity ligands may include, but are not limited to, one or more of: anti-CD19 antibody, anti-CD20 antibody, anti-CD21 antibody, anti-CD22 antibody, anti-sTn antibody, anti-CD5 antibody, Lym-1 antibody (antibody against the B cell determinant recognized by Lym-1; see, e.g., U.S. Pat. No. 5,789,554), CDIM antibody (antibody against the B cell determinant recognized by CDIM; see, e.g., U.S. Pat. No. 5,593,676), anti-CD45R (RAhi or RO) antibody, anti-Ki-M4 antibody, and anti-DRC-l antibody. An affinity ligand is used herein to also comprise an affinity ligand which has been coupled (using covalent or noncovalent or other means known in the art) to a detectable moiety. The term “detectable moiety” is used herein, for purposes of the specification and claims, to mean a label molecule that is directly or indirectly detectable and, as part of the affinity ligand, may be used to determine an amount of mononuclear cell phenotype in a sample. Detectable moieties may include, but not limited to, enzymes (e.g., peroxidase, alkaline phosphatase, etc.), radioisotopes, haptens (e.g., biotin, avidin, etc.), chromophores, fluorescent molecules, and fluorescent nanocrystals, as known to those skilled in the art of diagnostics. In a preferred embodiment, the detectable moiety comprises a fluorescent molecule comprising a fluorophore which may include, but is not limited to, fluorescein (isothiocyanate), fluorescein derivatives, pthalocyanine dyes, phycoerythrin, up-converting phosphors, peridinin-chlorophyll protein, fluorescamine, dansyl chloride, rhodamine, Texas red tandem, phycocyanin tandem, allo-phycocyanin tandem, and coumarin derivatives. The detectable moiety may be bound to a primary affinity ligand; or to a secondary affinity ligand which is then used to specifically bind to an unlabelled primary affinity ligand (e.g., a combination of primary antibody, and labeled secondary antibody).

The term “clinical sample” is used herein, for purposes of the specification and claims, to mean a fluid or tissue obtained from an individual; and in a preferred embodiment, is selected from the group consisting of peripheral blood, body fluids other than peripheral blood (particularly effusions associated with solid, non-lymphoid tumors), and lymphoid tissue. The term “clinical sample” also encompasses a preparation which is derived from the clinical sample, and which is enriched for mononuclear cells or for one or more subpopulation of mononuclear cells comprising mononuclear cell phenotype, as will be more apparent from the following descriptions. For example, the term “peripheral blood” also encompasses a preparation which is derived from peripheral blood, wherein the preparation is enriched for mononuclear cells or for one or more mononuclear cell subpopulations comprising mononuclear cell phenotype, as will be more apparent from the following descriptions.

The term “determinant” with reference to the mononuclear cell phenotype to be detected, is used herein, for purposes of the specification and claims, to mean a cell-associated molecule which may be used to detect and determine an amount of cells comprising mononuclear cell phenotype in a clinical sample; wherein each determinant is capable of binding to an affinity ligand having binding specificity and avidity for that determinant. As an illustrative example of a preferred embodiment, and as will be apparent to one skilled in the art from the following descriptions, a combination of determinants may be used to detect one or more mononuclear cell subpopulations comprising mononuclear phenotype. Determinants may include, but are not limited to, molecules (e.g., receptors, components, antigen, or shed tumor antigen, signaling molecules) present on the cell surface of one or more subpopulations comprising mononuclear cell phenotype, or molecules internal to such cells, or a combination thereof. In general, a determinant may be used alone or in combination with one or more other determinants to distinguish a particular mononuclear cell subpopulation comprising mononuclear cell phenotype from other subpopulations of cells which may be contained in the sample. In a preferred embodiment, the determinant may be selected from the group consisting of a pan B cell marker (e.g., CD19, CD20, CD72, and the like), a pan T cell marker (e.g., CD5 in absence of detectable CD19, and the like), a pan FDC marker (e.g., CD21 in absence of detectable CD19; K1-M4; DRC-l; HJ2; R4/23; FDC-M1; BU-10; CAN.42; and the like), and a marker expressed by mononuclear cells as a response to an immune process (e.g., CD21++, CD5 in presence of CD19, CD21 in the presence of CD19, an epitope comprising a terminal alpha 2,6 linked sialic acid (e.g., sTn), and a combination thereof. In a preferred embodiment, the determinant may be selected from the group consisting of CD19, CD21, CD5, sTn, and a combination thereof. As an illustrative example, a subpopulation comprising memory B cells (defined herein as CD5− B cells comprising mature B cells, or antigen-stimulated B lymphocytes, or progeny thereof other than plasma cells) comprising a mononuclear cell phenotype can be detected by detecting a combination of at least two determinants, wherein a first determinant comprises a pan B cell marker (e.g., CD19, CD20, CD72, and the like), and a second determinant comprises a marker for memory B cells (e.g., CD21, CD79 (a or b), CD75 (e.g., CDw75), CD45R (CD45RAhi or CD45RO) and CD22). A third determinant comprising a marker expressed by mononuclear cells, comprising an epitope comprising a terminal alpha 2,6 linked sialic acid (e.g., sTn), may be used. As an illustrative example, a subpopulation comprising B1 cells comprising a mononuclear cell phenotype can be detected by a combination of at least two determinants, wherein a first determinant comprises a pan B cell marker (e.g., CD19, CD20, CD72, and the like), and a second determinant comprises a mononuclear cell marker for B1 cells (e.g., CD5). A third determinant comprising a marker expressed by mononuclear cells, comprising an epitope comprising a terminal alpha 2,6 linked sialic acid (e.g., sTn), may also be used. As an illustrative example, a subpopulation comprising T cells comprising a mononuclear cell phenotype can be detected by a combination of at least two determinants, wherein a first determinant comprises a pan T cell marker (e.g., CD3, or CD5 in absence of detectable CD19, or functional equivalent), and a second determinant comprising a marker expressed by mononuclear cells, comprising an epitope comprising a terminal alpha 2,6 linked sialic acid (e.g., sTn). As an illustrative example, a subpopulation comprising follicular dendritic cells comprising a mononuclear cell phenotype can be detected by a combination of at least two determinants, wherein a first determinant comprises a pan FDC marker (e.g., CD21 in absence of detectable CD19; Ki-M4; DRC-1; HJ2; R4/23; FDC-M1; BU-10; or CAN.42), and a second determinant comprising a marker expressed by mononuclear cells, comprising an epitope comprising a terminal alpha 2,6 linked sialic acid (e.g., sTn).

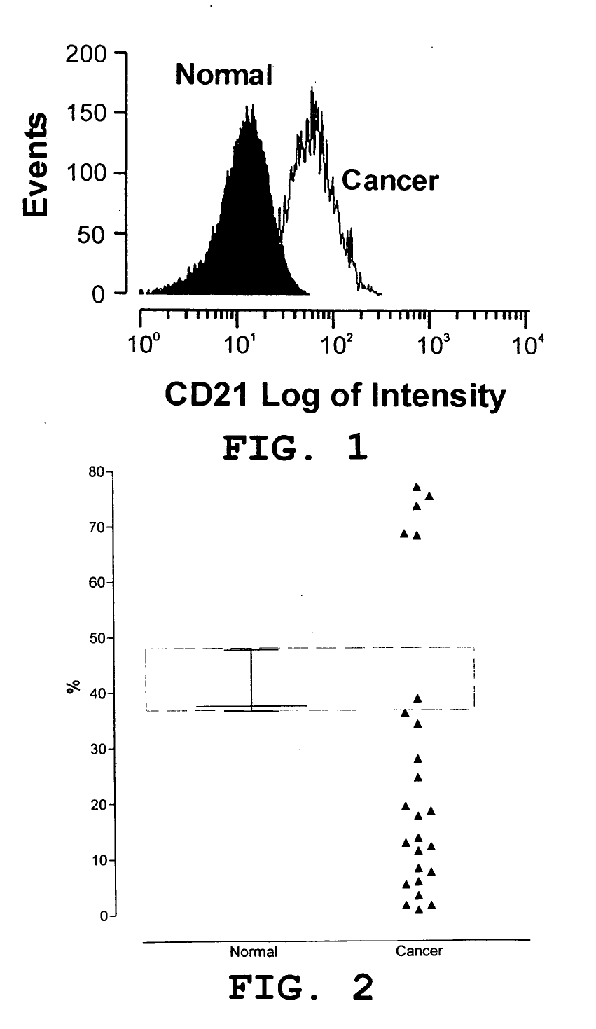

The term “CD21++” is used herein, for purposes of the specification and claims, to mean a hyperexpression of CD21 as compared to the amount of CD21 normally detected as present on B cells (“CD21+”). For purposes of illustration, CD21++ comprises a relative cell expression of CD21 which is equal to or greater than 3 times the normal relative B cell expression of CD21 (CD21+). For example, as measured by flow cytometry and in plotting the log intensity of CD21 staining of CD19+ cells, the average CD21 staining intensity of CD19+ cells of lymphoid tissue origin from individuals having tumor and a pro-tumor immune response was a relative value of about 65; whereas the average CD21 staining intensity of CD19+ cells of lymphoid tissue origin from control individuals (not having a pro-tumor immune response) was a relative value of about 15. In continuing this example, CD21++was distinguished from CD21+ cells by setting as a lower limit of the range of CD21++ a value which is higher than 95% of the CD21+ values as expressed by CD21+ B cells of healthy donors (see FIG. 1).

The term “individual” is used herein, for purposes of the specification and claims, to mean a mammal, and preferably a human, and more preferably a human who is being screened for, or at risk of developing, or has developed, a pro-tumor immune response. This may include an individual having a primary tumor comprising a solid, non-lymphoid tumor and/or its metastases; an individual having a pre-cancerous lesion comprising transformed (abnormal in proliferation and/or genetic makeup as compared to normal epithelial cells of the same type) cells of epithelial origin which release shed tumor antigen; an individual who is at high risk (e.g., environmentally and/or genetically) for developing a solid, non-lymphoid tumor; or an individual who has been treated for a solid, non-lymphoid tumor and thereby inherently carries a risk of recurrence. A method according to the present invention is to screen for a pro-tumor immune response in such an individual at risk for developing, or who has developed, a pro-tumor immune response by detecting the presence or absence of an indicator comprising an alteration in mononuclear cell phenotype. The presence of a pro-tumor immune response may also be an indicator for either tumor progression, or for susceptibility to tumor development.

The term “lymphoid tissue” is used herein, for purposes of the specification and claims, to mean a tissue which contains localized areas of antigen presenting cells (e.g., follicular or germinal center dendritic cells) and B lymphocytes, and in which can be induced an immune response involving B cells. An example of such localized areas comprises germinal centers. Such lymphoid tissues comprise lymphatic tissues including, but not limited to, lymph nodes; milky patches in the mesenterium of the intestine; omentum; appendix; Peyer's patches; loose connective tissue (e.g., associated with vessels in the walls of the aorta); lymphatic vessels; submucosal spaces; subserosa spaces; peritoneal cavity; ligaments (e.g., gastrohepatic ligament); and epineura. “Lymphoid tissue” is inclusive of lymphoid tissues infiltrated with shed tumor antigen, which may become involved in a reactive process which includes an expansion in the size of germinal centers or germinal center equivalents, and an infiltration and/or proliferation of B cells, particularly memory B cells and other B cells activated by shed tumor antigen. Generally, such lymphoid tissues may be found regional (draining) or distal to a primary tumor or its metastases. The term “lymphoid tissue” when used in reference as a sample from which mononuclear cell phenotype is determined, also encompasses a preparation which is derived from the lymphoid tissue, and which is enriched for mononuclear cells or a subpopulation thereof, as will be more apparent from the following descriptions.

The term “amount” is used herein, for purposes of the specification and claims, a number which is expressive of a quantity of mononuclear cell phenotype determined from a clinical sample (or determined from one or more samples for obtaining a reference value). For example, the amount of mononuclear cell phenotype from the determination may be expressed as the actual (e.g., absolute) number of mononuclear cells of that phenotype by itself. Alternatively, amount from the determination may be expressed in relation to a certain parameter (as a relative value); e.g., number of mononuclear cells of that phenotype in relation to the quantity of blood or fluid (e.g., number of cells/ml), or number of mononuclear cells of that phenotype in relation to the number of a total cell population (e.g., percentage of the number of total white blood cells, or percentage of the number of overall B cells (e.g., where a B cell subpopulation is determined), or percentage of the number of overall T cells (e.g., where a T cell subpopulation is determined), or percentage of the number of total lymphocytes (where a B cell subpopulation or T cell subpopulation is determined), or percentage of number of mononuclear cells), or number of mononuclear cells of that phenotype in relation to a reference value (e.g., in relation to a predetermined clinical value).

The term “difference” is used herein relative to a reference value, for purposes of the specification and claims, to mean that the amount of the mononuclear cell phenotype determined from analysis of a test sample falls outside the range of normal clinical values for that phenotype that is established by prospective and/or retrospective statistical clinical studies, or differs from the amount of the mononuclear cell phenotype determined from a previous clinical sample (of the same sample type obtained from the same individual). As apparent to one skilled in the art, to be able to compare the value from the test sample with the reference value, the amounts of the same mononuclear cell subpopulations must be determined (e.g., the amount of sTn+ B cells determined from analysis of the test sample is compared to a reference value for sTn+ B cells). As also apparent to one skilled in the art, a “reference value” will depend on the type of comparison to be made. For example, for prognostic use the reference value may comprise an earlier value for that sample type from the same individual when looking for a change in the amount of mononuclear cell phenotype since the last determination and/or after treatment; and for diagnostic use, the reference value may comprise a range of normal clinical values for that sample type. In either case, the test sample from which an amount of mononuclear cell phenotype is determined should also be of the same sample type from which the reference value is determined (e.g., an amount of mononuclear cell phenotype from a test sample comprising peripheral blood should be compared to a reference value obtained from analysis of peripheral blood). A difference in amount of the mononuclear cell phenotype from a test sample, when compared to a reference value comprising a range of normal clinical values for that phenotype, comprises an alteration in mononuclear cell phenotype. An alteration in mononuclear cell phenotype may be an indicator of a pathological condition (e.g., solid, non-lymphoid tumor and a pro-tumor immune response, or a pro-tumor immune response). In a preferred use, the term “difference” is used herein, for purposes of the specification and claims, to mean that there is a statistically significant difference between an amount of the mononuclear cell phenotype determined from analysis of a test sample (“first value”) and an amount of mononuclear cell phenotype comprising a reference value to which it is compared (“second value”). For example, a first value that is statistically significant different as compared to a second value may comprise the first value being a number that is at least about two standard deviations outside the mean of the second value. In one example, the first value and the second value may be obtained from the same individual at different points in time (e.g., to monitor the course (state) of the pathological condition, or to test the efficacy of treatment of the pathological condition) in obtaining an indicator comprising a “prognostic value”. In another example, the first value is determined from a clinical sample obtained from an individual being screened for a pro-tumor immune response, and a second value is a predetermined reference value (e.g., determined from analyses of samples from individuals lacking a pro-tumor immune response, such as apparently healthy individuals), in obtaining an indicator comprising a “diagnostic value”. A statistically significant difference between an amount of a mononuclear cell phenotype in an individual having a pro-tumor immune response as compared to the amount comprising a reference value may be a difference represented by: V_{rd}>Mean+2.5(SEM), wherein “V_{rd}” is a value comprising an amount of the mononuclear cell phenotype determined from analysis of a clinical sample from an individual having a pro-tumor immune response that is above the reference value (expressed as a Mean plus 2.5 times the standard error of the Mean); or by V_{rd}<Mean−2.5(SEM), where the amount of the mononuclear cell phenotype determined from an individual having a pro-tumor immune response is below the reference value, as will be more apparent from the following embodiments. In general, the methods of the present invention are used to generate indicators to identify individuals as having or lacking a pathological condition, in providing an additional parameter to a competent health professional in making a medical opinion.

The term “solid, non-lymphoid tumor” is used herein, for purposes of the specification and claims, to mean any primary tumor of ductal epithelial cell origin, including, but not limited to, tumors originating in the liver, lung, brain, bone marrow, breast, colon, pancreas, stomach, rectum, prostate, or reproductive tract (e.g., cervix, ovaries, endometrium etc.); and which produces shed tumor antigen (e.g., serous, or endometroid, or mucinous tumors). For purposes of the present invention, “solid, non-lymphoid tumor” may also include a melanoma which produces shed tumor antigen.

The term “shed tumor antigen” is used herein, for purposes of the specification and claims, to mean a glycomolecule (e.g., glycoprotein or glycolipid) which:
(a) by itself, or in an aggregated or oligomeric (two or more monomers which are together) form, has a molecular size equal to or greater than about 100 kilodaltons;
(b) is released (e.g., shed) from a primary non-lymphoid tumor or its metastases (“primary source”), thereby becoming soluble and allowing movement into lymphoid tissues regional or distal to the primary source;
(c) comprises a molecule which comprises a carbohydrate epitope present more than once on the molecule (e.g., the molecule has a plurality of carbohydrate chains, wherein several of the carbohydrate chains express the same carbohydrate epitope; hence the carbohydrate epitope is repeated in the structure of the molecule), wherein the carbohydrate epitope comprises one or more of: Tn antigen (comprising a terminal N-acetyl galactosamine), or a terminal 2,6 linked sialic acid (e.g., sTn antigen comprising a terminal sialic acid 2,6-linked to N-acetyl galactosamine; or a terminal sialic acid 2,6-linked to galactose), the structures of which are known in the art;
(d) is capable of inducing a humoral immune response, which may ultimately result in the production and secretion of anti-shed tumor antigen antibody which is predominately of an IgG class; and
(e) can interact with anti-shed tumor antigen antibody in forming immune complexes, wherein the immune complexes may bind and crosslink Fc receptors (FcR) present on the surface of FcR-expressing cells.

For purposes of illustration, and not limitation, exemplifying such shed tumor antigen are mucins (e.g., the glycoprotein encoded by the MUC-1 gene) and mucin-like molecules (e.g., carcinoembryonic antigen (CEA), Sialyl Lewis a, and the like) produced and shed by solid, non-lymphoid tumor. For purposes of illustration, and not limitation, in a preferred embodiment of the present invention, the shed tumor antigen comprises the gene product of the MUC-1 gene (also known as polymorphic epithelial mucin). Shed tumor antigen and anti-shed tumor antigen antibodies may form immune complexes that may have a threshold level for spacing and number of antibody molecules necessary for Fc receptor (e.g., FcγRI) crosslinking.

The term “pro-tumor immune response”, for purposes of the specification and claims, means a humoral immune response against a terminal, carbohydrate epitope of shed tumor antigen resulting in the production of antibody (particularly IgG) to shed tumor antigen, wherein the antibody binds shed tumor antigen in forming immune complexes. Such immune complexes may promote tumor progression (one or more of tumor growth, invasion, or metastasis) by one or more mechanisms including, but not limited to, binding and cross-linking Fc receptors (FcR; e.g., FcγRI) on immune effector cells resulting in the release of inflammatory mediators which promote local tissue destruction and angiogenesis; and binding and crosslinking receptors expressed on endothelial cells resulting in an induction of endothelial cell proliferation and/or release of factors promoting angiogenesis. Immune effector cells are host cells which are mediators of inflammation and/or angiogenesis (e.g., one or more of granulocytes, macrophages, vascular endothelial cells) that are capable of inducing a cascade of processes which promote tumor progression. For example, after activation by such immune complexes, granulocytes and macrophages cooperate to release tissue degradative enzymes which breakdown the connective tissue matrix, thereby facilitating invasion of the tumor and spread of metastases beyond the primary tumor.

Measurement and quantitation of cell subpopulations in a clinical sample obtained from an individual can be important in assessing certain pathological conditions. Direct measurement of such subpopulations may provide an accurate assessment of the condition of, or susceptibility to, disease in the individual at the time the sample is taken. For example, the number of CD4+ T helper cells in peripheral blood has been used as an indicator of progression of HIV infection, and for monitoring treatment of the disease, in an individual. However, currently the prognosis of a tumor-bearing individual who undergoes anticancer therapy (one or more of surgery, chemotherapy, immunotherapy, photodynamic therapy, radiotherapy, and the like) is mainly determined by the extent of residual tumor load, comprising either primary tumor and/or presence of micro-metastases (occult to current imaging techniques), following anticancer therapy. While detection of primary tumor cells and metastatic tumor cells may provide information clinically significant to the tumor bearing individual, the presence of such cells may not be an accurate predictor of further tumor development (recurrence), nor an accurate predictor if the individual can mount, or has mounted, an effective antitumor immune response. Further, presently there are no commercially available tests to evaluate for the presence of a pro-tumor immune response. There is a need for laboratory tests that distinguish an individual whom is more likely to have a favorable prognosis (e.g., one or more of stable remission; limited, localized disease progression; response to anticancer therapy that reduces the rate of recurrence of cancer) from an individual whom is likely to have an unfavorable prognosis (e.g., an individual having undergone anti-cancer therapy but whom still has indications of a pro-tumor immune response, and is at risk for recurrence; an individual having both tumor and a pro-tumor immune response; or an individual whom has a pro-tumor immune response that is advancing/progressing).

In that regard, the present invention relates to a discovery that alteration in mononuclear cell phenotype can be present in individuals having tumor and a pro-tumor immune response, and in individuals having a pro-tumor immune response (e.g., after removal or reduction of substantially all tumor mass; or during a pre-cancerous condition such as before detectable tumor). In a preferred embodiment of the present invention, an indicator for the presence of a pro-tumor immune response may comprise detection of alteration in mononuclear cell phenotype in one or more of (a) peripheral blood, (b) body fluids other than peripheral blood (particularly cell-containing effusions associated with solid, non-lymphoid tumors), or (c) lymphoid tissues containing deposits of shed tumor antigen. Thus, in the diagnostic methods and prognostic methods of the present invention, a clinical sample is assayed to determine an amount of mononuclear cell phenotype, and whether the amount of mononuclear cell phenotype determined is different (e.g., increased or decreased) with respect to a reference value for the mononuclear cell phenotype.

In one embodiment of the present invention, an alteration in mononuclear cell phenotype that comprises an indicator (comprising a diagnostic value) for the presence of a pro-tumor immune response (tumor and a pro-tumor immune response, or a pro-tumor immune response in absence of detectable tumor) in an individual may comprise a determination of a significant decrease in an amount of the overall subpopulation of B cells in the peripheral blood (e.g., B cells measured by using a pan B cell marker) as compared to a reference value (comprising a normal range of clinical values), combined with an alteration in a mononuclear cell subpopulation other than overall B cells. As one illustrative, non-limiting but preferred example, an alteration in mononuclear cell phenotype comprising the indicator may comprise a decrease in amount of peripheral blood B cells, in combination with an increase in an amount of one or more B cell subpopulations (e.g., sTn+ B cells, memory or mature B cells (e.g. CD19+CD21+ cells), CD21++ memory or mature B cells, sTn+ B1 cells (e.g., CD19+CD5+sTn+ cells)) determined from a sample comprising peripheral blood and which amount is different compared to the reference value for the respective one or more B cell subpopulations.

Alternatively, the indicator may comprise an alteration in one or more mononuclear cell subpopulations, other than an overall B cell type subpopulation or an overall FDC subpopulation in lymphoid tissue, comprising mononuclear cell phenotype. As a preferred illustration, an alteration in mononuclear cell phenotype comprising the indicator may comprise an increase in an amount (with respect to a respective reference value) of a mononuclear cell subpopulation selected from the group consisting of sTn+ T cells, overall FDC in peripheral blood, sTn+ FDC, sTn+ B cells (e.g., CD19+sTn+ cells), memory B cells (e.g., CD19+CD21+ cells), CD21++ memory B cells (e.g., CD19+CD21++ cells), sTn+ B1 cells (e.g., CD19+CD5+sTn+ cells), and a combination thereof. Such an indicator may further comprise a decrease in an amount (with respect to a respective reference value) of a peripheral blood mononuclear cell subpopulation selected from the group consisting of sTn+ memory B cells (e.g., CD19+CD21+sTn+ cells). A preferred alteration in mononuclear cell phenotype may be determined, and used as an indicator, to the exclusion of alteration in mononuclear cell phenotype other than the preferred alteration in mononuclear cell phenotype.

In another example, an indicator comprising a prognostic value for an individual who has had substantially all or a majority of tumor mass removed or reduced by anticancer therapy comprises determining an amount, in each of successive samples (e.g., one or more samples pre-anticancer therapy, and one or more samples post-anticancer therapy) from the individual, of one or more mononuclear cell subpopulations comprising mononuclear cell phenotype. By comparing the effect of anticancer therapy on the amount of the mononuclear cell phenotype, prognostic information or information relating to the efficacy of the therapy (including possible need for modification of the treatment regimen) may be obtained. As an illustrative example, an individual undergoing anticancer therapy, as determined from one or more samples post-anticancer therapy, may show one of several patterns of alteration in mononuclear cell phenotype. In one illustration, wherein a pro-tumor immune response still exists after anticancer therapy, detected in a body fluid (preferably peripheral blood) sample obtained from the individual post-treatment may be a continuing presence of an alteration in mononuclear cell phenotype. The alteration in mononuclear cell phenotype may be compared to a reference value, wherein the reference value is selected from the group consisting of a normal range of clinical values, a value obtained from the same individual pre-treatment, a value obtained from the same individual during treatment but before conclusion of treatment, and a combination thereof. For example, for purposes of illustration, the alteration in mononuclear cell phenotype may comprise a difference in a mononuclear cell subpopulation (expressed as an amount relative to the respective reference value comprising a normal range of clinical values for the subpopulation determined) selected from the group consisting of: an increase in sTn+ B cells, an increase in CD21+ B cells, an increase in CD21++ B cells, a decrease in CD21+sTn+ B cells, an increase in sTn+ B1 cells, an increase in sTn+ T cells, an increase in sTn+ follicular dendritic cells, an increase in peripheral blood FDC, a decrease in sTn+CD21+ B cells (e.g., CD19+CD21+sTn+ cells), and a combination thereof. The alteration in mononuclear cell phenotype may further comprise a decrease in an overall B cell population (e.g., in CD19+ cells). Such an alteration in mononuclear cell phenotype may be an indicator of prognostic value that anticancer therapy was ineffective in substantially reducing the pro-tumor immune response. Thus, the individual may have an increased risk of recurrence due to the continued presence of the pro-tumor immune response (hence, a prognostic value); and may be a candidate for further therapy that is targeted to substantially reducing the pro-tumor immune response. A preferred alteration in mononuclear cell phenotype may be determined and used as a prognostic indicator to the exclusion of an alteration in phenotype other than the preferred alteration in phenotype.

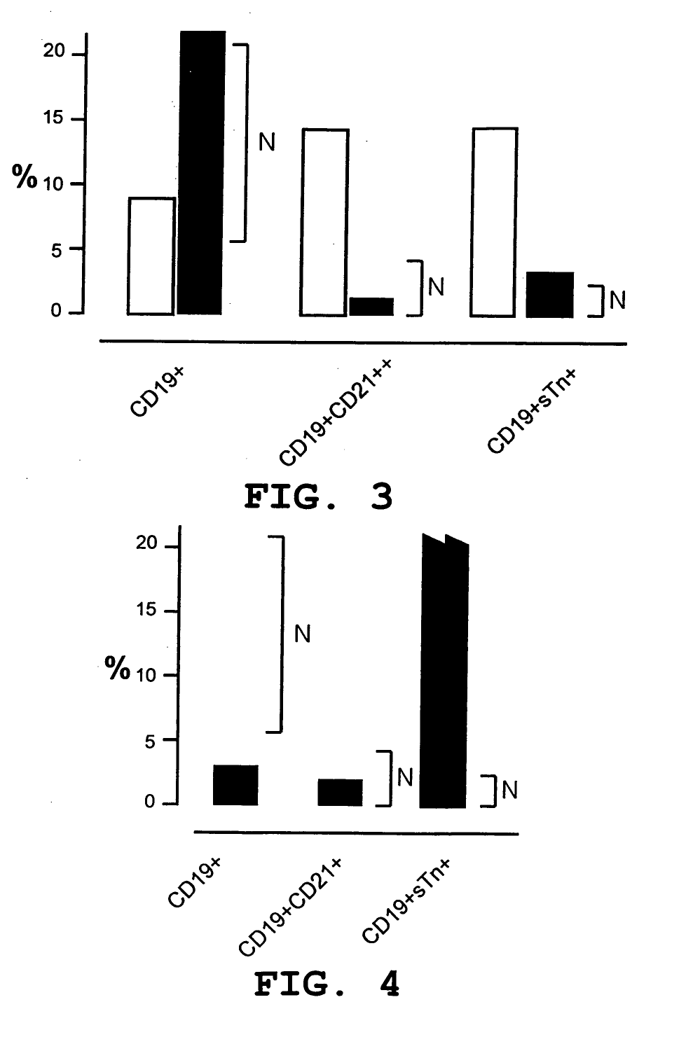

In an illustrative example wherein substantial reduction of a pro-tumor immune response (either with substantial reduction of tumor, or reduction of a pro-tumor immune response alone) has been achieved, detected in a clinical sample obtained post-anticancer therapy from the individual is a difference in an amount of mononuclear cell phenotype (“second value”) as compared to an amount determined for the mononuclear cell phenotype from the same type of clinical sample wherein the sample was obtained from the same individual before anticancer therapy was begun or before it is concluded (“first value”). In continuing this embodiment, the second value shows a difference when compared to the first value; i.e. the second value approaches or falls within the reference value (comprising a normal range of clinical values) for the mononuclear cell phenotype (see, e.g., Example 4, and FIG. 3, herein). Detection of such a change in amount of mononuclear cell phenotype as a result of anticancer therapy may be indicative of the individual's positive response to anticancer therapy, and may also be an indicator that this individual has a reduced chance of recurrence as compared to an individual who demonstrates no significant difference in (e.g., where the second value does not differ from the first value), or demonstrates a worsening of the pro-tumor immune response subsequent to anticancer therapy (e.g., as demonstrated by the second value deviating farther outside the reference value (comprising a normal range of clinical values) than the first value).

In accordance with one embodiment of the method for screening for a pro-tumor immune response according to the present invention, the method comprises: (a) contacting a clinical sample, obtained from the individual, with one or more affinity ligands for determining the amount of (e.g., by detecting and quantifying an amount of cells in the sample which are bound by the one or more affinity ligands), mononuclear cell phenotype in the sample; and (b) comparing the amount of mononuclear cell phenotype determined in the sample to a reference value for the mononuclear cell phenotype; wherein a difference in the amount of mononuclear cell phenotype determined as compared to the reference value may be an indicator of the presence of a pro-tumor immune response. Preferably, substantially the same methodology used to determine the mononuclear cell phenotype from the sample being screened is used to determine the mononuclear cell phenotype comprising the reference interval. A difference in amount of mononuclear cell phenotype determined (when compared to a reference value comprising a normal range of clinical values) comprises an alteration in mononuclear cell phenotype. In a preferred embodiment, when the clinical sample comprises a body fluid such as peripheral blood, the alteration in mononuclear cell phenotype may comprise a subpopulation selected from the group consisting of (expressed as an amount relative to the reference value for the mononuclear cell subpopulation determined): an increase in sTn+ B cells, an increase in CD21+ B cells, an increase in CD21++ B cells, a decrease in CD21+sTn+ B cells, an increase in sTn+ B1 cells, an increase in sTn+ T cells, an increase in sTn+ follicular dendritic cells, a increase in overall follicular dendritic cells, and a combination thereof. The altered mononuclear cell phenotype may further comprise a decrease in overall B cells (e.g., CD19+ cells). In another preferred embodiment in which the clinical sample comprises lymphoid tissue, the alteration in mononuclear cell phenotype may comprise a mononuclear cell subpopulation selected from the group consisting of (expressed as an amount relative to the reference value for the mononuclear cell subpopulation determined): an increase in sTn+ B cells, an increase in CD21+ B cells, an increase in CD21++ B cells, an increase in sTn+ follicular dendritic cells, and a combination thereof. The altered mononuclear cell phenotype may further comprise an increase in overall B cells (e.g., CD19+ cells), an increase in overall FDC, and a combination thereof. A preferred alteration in mononuclear cell phenotype may be determined and used as an indicator to the exclusion of alteration in mononuclear cell phenotype other than the preferred alteration in mononuclear cell phenotype.

In accordance with another embodiment of the method according to the present invention, the method comprises determining the state or status (e.g., progression or advancement, or reduction, or no change, in the course of) a pro-tumor immune response, at a certain point in time, in an individual having a pro-tumor immune response (either in the presence or absence of detectable tumor) by determining an amount of mononuclear cell phenotype in each of successive samples obtained from the individual. Hence, the status of the pro-tumor immune response in the individual is determined by comparing an amount of the mononuclear cell phenotype in a sample obtained from the individual for establishing a reference value for that particular individual's pro-tumor immune response (e.g., “reference sample”) to an amount of mononuclear cell phenotype in a sample obtained from the individual subsequent to the reference sample (“test sample”) in determining the state of the pro-tumor immune response at the time at which the test sample was obtained from the individual. The reference sample and test sample may be analyzed for their respective amount of mononuclear cell phenotype using essentially the same methodology for measuring the amount of the same one or more mononuclear cell subpopulations comprising the mononuclear cell phenotype (see, for example, FIGS. 5-8). Preferably the reference sample and test sample comprise the same sample type; e.g., each sample comprises a sample of peripheral blood or each sample comprises a sample of lymphoid tissue. In illustrating this embodiment, the method comprises: (a) contacting a clinical sample comprising a test sample, obtained from the individual, with one or more affinity ligands for determining the amount of (e.g., by detecting and quantifying an amount of cells in the test sample which are bound by the one or more affinity ligands), mononuclear cell phenotype in the test sample; and (b) comparing the amount of mononuclear cell phenotype determined in the test sample to an amount of mononuclear cell phenotype determined in a reference sample obtained from the individual; wherein presence or absence of a difference between the amount of mononuclear cell phenotype in the test sample and the amount of mononuclear cell phenotype in the reference sample comprises an indicator for the status of the pro-tumor immune response at a time at which the test sample was obtained from the individual. For example, when an amount of mononuclear cell phenotype determined from test sample is the same, or approximately the same (e.g., no appreciable difference; i.e., differs only within the variance established for the method used for the determination), as the amount of mononuclear cell phenotype determined from the reference sample, such a result may be an indicator that the status of the individual's pro-tumor immune response is unchanged during the time period between the time at which the reference sample was obtained and the time at which the test sample was obtained. However, where the amount of mononuclear cell phenotype determined from test sample deviates farther away from (e.g., outside of) a normal range of clinical values than does the amount of mononuclear cell phenotype determined from the reference sample, such a result is an indicator of a status in which the pro-tumor immune response has advanced (e.g., the pro-tumor immune response has progressed to a more pathological condition) during the time period between the time at which the reference sample was obtained and the time at which the test sample was obtained. Alternately, where the amount of mononuclear cell phenotype determined from test sample shows a difference when compared to the amount of mononuclear cell phenotype determined from the reference sample, wherein the difference is that the amount determined from the test sample approaches (e.g., is closer than that of the reference value) or falls within a normal range of clinical values for that mononuclear cell phenotype, such a result is an indicator that the pro-tumor immune response has been reduced (e.g., the pro-tumor immune response has decreased in intensity or has been suppressed) during the time period between the time at which the reference sample was obtained and the time at which the test sample was obtained. As previously discussed in more detail herein in which the prognostic method is used to monitor efficacy of anticancer therapy, the reference sample is generally obtained from the individual before anticancer therapy is initiated or in the initial period of treatment (before the therapy is expected to show any clinical effects), and the test sample is generally obtained at a point in time after anticancer therapy has been administered to the individual (e.g., including at the conclusion or after the conclusion of a regimen of anticancer therapy). A preferred mononuclear cell phenotype may be used to determine the status of a pro-tumor immune response to the exclusion of mononuclear cell phenotype other than the preferred phenotype.

In a preferred embodiment of the methods according to the present invention, the one or more affinity ligands for determining an amount of mononuclear cell phenotype in a sample comprises a plurality of affinity ligands comprising:
(a) one or more first affinity ligands which specifically bind a determinant comprising a pan B cell marker, and one or more second affinity ligands which specifically bind a determinant comprising a determinant found on one or more B cell subpopulations (e.g., CD21, hyperexpressed CD21, CD5, or a terminal 2,6-linked sialic acid (linked to galactose or N-acetylgalactosamine; e.g., sTn);
(b) one or more one or more first affinity ligands which specifically bind a determinant comprising a pan T cell marker, and one or more second affinity ligands which specifically bind a determinant comprising an epitope comprising a terminal 2,6-linked sialic acid (e.g., sTn);
(c) one or more first affinity ligands which specifically bind a determinant comprising a pan follicular dendritic cell marker, and one or more second affinity ligands which specifically bind a determinant comprising an epitope comprising a terminal 2,6-linked sialic acid (e.g., sTn);
(d) and a combination thereof. As apparent to one skilled in the art from descriptions herein and as standard in the art, in this and other examples described herein an affinity ligand used in the method may comprise (e.g., be labeled with) a detectable moiety, or may comprise an unlabeled affinity ligand that is combined with a labeled secondary affinity ligand which has binding specificity for the unlabeled affinity ligand.

In another preferred embodiment, the plurality of affinity ligands is used to determine a mononuclear cell phenotype comprising amounts of B cells, T cells, and follicular dendritic cells. The combination of affinity ligands may comprise a first affinity ligand which specifically binds a determinant comprising CD19, a second affinity ligand which specifically binds a determinant comprising CD21, a third affinity ligand which specifically binds a determinant comprising CD5, and a fourth affinity ligand which specifically binds a determinant comprising sTn.

Additionally, test kits are provided for determining an amount of mononuclear cell phenotype in a clinical sample. Since we have devised several methods for treating a pro-tumor immune response in an individual, by detecting a pro-tumor immune response in an individual, various treatment options may be made available to the individual.

For purposes of the description, the methods and compositions of the present invention will be illustrated in the following examples.

### EXAMPLE 1

This Example illustrates that a clinical sample to be tested for an amount of mononuclear cell phenotype may be used as obtained, or may processed in a manner that includes, but is not limited to, enrichment for mononuclear cells (e.g., containing lymphocyte populations such as T cells and B cells, and containing follicular dendritic cells), and enrichment for lymphocyte subpopulation (e.g., B cells, or T cells or B cells and T cells). In one embodiment, the clinical sample is used as obtained (i.e., is not enriched for mononuclear cells). For example, a sample of whole blood was collected by venipuncture of an individual, wherein the blood was collected in a heparinized collection tube. An aliquot of the blood was directly analyzed for determining an amount of mononuclear cell phenotype by incubating the aliquot (e.g., 100 μl) with an amount of the one or more affinity ligands (added at an amount and dilution recommended by the manufacturer/supplier of the affinity ligand), and then the mixture was processed in an automated sample processor (e.g., in which red blood cells may be lysed, a cell stabilizer solution may be added, and a cell fixative may be added, and a combination thereof). While any suitable automated cell processor may be used, in this example the automated cell processor comprised a Coulter TQ-PREP (Beckman Coulter). After processing, the processed aliquot was analyzed by flow cytometry (using methodology described herein in more detail).

Methods of enriching a clinical sample, such as a sample of peripheral blood, for mononuclear cells are well known in the art. For example, mononuclear cells may be isolated from a clinical sample by overlaying the sample on a density gradient medium (e.g., Ficoll-Hypaque or Percoll or Lymphocyte Separation Medium) and then performing density gradient centrifugation. Depending on the density gradient medium used, typically the mononuclear cells may be harvested from the interface or buffy layer of the gradient.

The mononuclear cell population may be further processed to obtain a cell subpopulation enriched in B cells using one of several methods known to those skilled in the art. For example, neuramimidase-treated sheep red blood cells may be added to the mononuclear cell population, and the mixture may be centrifuged in a density gradient medium. T cells will bind (rosette) with the sheep red blood cells, and therefore are found in the cell pellet. In contrast, a lymphocyte subpopulation enriched in B cells would remain at the interface and can be harvested. Alternatively, a lymphocyte subpopulation enriched in B cells may be obtained in a negative selection process. For example, the mononuclear cell population may be mixed with magnetic beads coated with one or more antibodies that bind to T lymphocytes (e.g., anti-CD2 mAb, anti-CD3 mAb, anti-CD4 mAb, anti-CD8 mAb, anti-CD28 mAb, or a combination thereof). A magnetic field is then applied, thereby immobilizing the T lymphocytes. The rest of the cell suspension (portion of the mononuclear cells which are not immmobilized) comprise a lymphocyte subpopulation enriched in B cells. The T cells may be eluted from the magnetic beads using methods known in the art; hence resulting in a positive selection and a preparation comprising a lymphocyte subpopulation enriched in T cells.

In a method of positive selection, a lymphocyte subpopulation enriched in B cells may be obtained from the clinical sample. For example, the clinical sample is mixed with magnetic beads coated with antibodies that bind to most B lymphocytes (e.g., anti-CD19 mAb, anti-CD20 mAb). A magnetic field is then applied, thereby immobilizing the B lymphocytes. The rest of the cell suspension (portion of the mononuclear cells which are not immobilized) may be used as a preparation enriched in mononuclear cells selected from the group consisting of T cells, FDC, or a combination thereof. The magnetic beads-B cell complex may be applied directly in an assay for quantitating B cells, or the B cells may first be eluted from the magnetic beads using methods known to those skilled in the art (e.g., competition with free ligand).

Generally, most FDC float at densities greater than 1.06 g/ml on low density albumin gradient or Percoll gradient or Ficoll-Urografin gradient (Schnizlein et al., 1985, J. Immunol. 134: 1360-8; Petrasch et al., 1990, Eur. J. Immunol. 20: 1013-8). A mononuclear cell population may be further processed to obtain a cell subpopulation enriched in FDC using one of several methods known to those skilled in the art. For example, opsonized sheep red blood cells may be added to the mononuclear cell population, and the mixture may be centrifuged in a density gradient medium. FDC will form rosettes with opsonized sheep red blood cells, and therefore are found in the cell pellet. Alternatively, a subpopulation enriched for FDC may be obtained in a double selection process. For example, the mononuclear cell population may be mixed with magnetic beads coated with one or more antibodies that bind to CD19+ cells. A magnetic field is then applied, thereby immobilizing populations of cells comprising B lymphocytes. The rest of the cell suspension (portion of the mononuclear cells which are not immmobilized) is removed, and then mixed with magnetic beads coated with one or more antibodies that bind to CD21+ cells. A magnetic field is then applied, thereby immobilizing populations of cells substantially comprising FDC (CD21+). The magnetic beads-FDC complex may be applied directly in an assay for quantitating FDC, or the FDC may first be eluted from the magnetic beads using methods known to those skilled in the art (e.g., competition with free ligand). Also, discontinuous gradient centrifugation and magnetic separation may be used in combination to isolate FDC. For example, mononuclear cells can be layered onto a discontinuous bovine albumin gradient, followed by centrifugation at 8500×g. The cells suspended at the 1.052 to 1.030 interphase are collected. Such cells are incubated with biotin labelled KiM4 mAb, then attached to streptavidin-conjugated paramagnetic beads, and then sorted using a magnetic sorter, thereby resulting in an average FDC content of 78% (Schmitz et al., 1993, J. Immunol. Methods 26: 189-196). Alternatively, flow cytometric cell sorting may be performed to isolate HJ2+ FDC.

### EXAMPLE 2

This Example illustrates assaying a clinical sample in determining an amount of mononuclear cell phenotype contained therein. As apparent to those skilled in the art from the descriptions herein, the assay can be performed by a process that includes, but is not limited to, immunofluorescence, chemiluminescence, flow cytometry, and a cell-based assay such as a cell-based enzyme-linked immunosorbent assay (“cELISA”). In a cELISA, the cells prepared from the clinical sample, which are to be assay for the mononuclear cell phenotype to be determined, are fixed to the wells of an ELISA plate. For example, each well of a 96 well plate may be incubated with a 100 μl of a solution of poly-L-lysine hydrobromide (5 mg/ml) in a buffered saline solution for 30 minutes at room temperature. After removing the solution, the cells (about 100,000 to 200,000/well) are plated, the plate is then centrifuged (e.g., at 100 g for 5 minutes), and the supernatant is then removed. Glutaraldehyde in buffer (0.25%) is added to each well, and then incubated for 5 minutes at room temperature. The wells containing fixed cells may then be washed with a Tris-buffered saline or other suitable solution, and affinity ligands may then be added in accordance with an ELISA protocol in assaying for the mononuclear cell phenotype to be determined (e.g., incubation with one or more affinity ligands, one or more washes, and subsequent detection of the one or more affinity ligands bound to the fixed cells). As will be apparent to one skilled in the art, an amount of the mononuclear cell phenotype may be determined and then expressed in relation to another parameter, as previously described herein in more detail. However, it will be apparent to one skilled in the art that the amount determined of a mononuclear cell phenotype may vary depending on factors which include, but are not limited to, the specific processes (methodology) used to determine the amount of mononuclear cell phenotype, the nature of the affinity ligands used in the determination, the origin and processing of the clinical sample assayed for the mononuclear cell phenotype, and the laboratory personnel and instruments used to perform the assays.

In a preferred and illustrative embodiment, flow cytometry is used to determine an amount of the mononuclear cell phenotype. The general principles involved in flow cytometry are well known in the art. Briefly, parameters that may be used in the assay include light scatter (e.g., to gate on one or more mononuclear cell subpopulations based on size, granularity and cell volume), fluorescence emission spectra and intensity thereof (to determine which affinity ligands are bound and, hence which cells are present; and the amount of expression by a cell of the determinant bound by affinity ligand, and a number of cells in a sample which express that determinant). It is known to those skilled in the art that flow cytometry can detect cells specifically bound by affinity ligands labeled with more than one type of fluorescent molecule.

In one embodiment of determining an amount of mononuclear cell phenotype in a clinical sample, mononuclear cells were isolated from the clinical sample using a density gradient medium and by density gradient centrifugation. Aliquots of the sample, each aliquot containing approximately 1 million cells, were treated in one of several different ways. A first aliquot of cells was left unstained, so as to act as a control for possible auto-fluorescence. A second aliquot of cells was mixed in a staining process with isotype affinity ligand. To illustrate this point, it is widely accepted by those skilled in the art that a desirable control for setting the negative region markers (to account for the fluorescence due to non-specific background observed with the staining process) is to stain with a mAb of the same subclass as the mAb used in the testing, but with an irrelevant specificity (e.g., does not specifically recognize a determinant on the cells of the mononuclear cell phenotype to be determined), and is commonly referred to as an “isotype control”. Thus, the second aliquot was mixed with an IgG1 control antibody labeled with FITC, an IgG1 control antibody labeled with Pe, and an IgG1 control antibody labeled with Pe-Cy5. This treated second aliquot serves as a negative control relative to any non-specific binding of the isotype (IgG1) antibodies to the cells to be detected. A third aliquot of cells may be stained with one or more affinity ligands having binding specificity for the mononuclear cell phenotype to be determined. For example, and in continuing with this exemplary embodiment, the third aliquot of cells is double-stained (stained jointly) with anti-CD19 antibody (IgG1 mAb) labeled with Pe-Cy5, and an anti-CD21 antibody (IgG1 mAb) labeled with FITC. Additional aliquots may be stained with other combinations of affinity ligands having binding specificity for cells of the mononuclear cell phenotype to be detected. For purposes of illustration, but not limitation, one such other combination is triple-staining of an aliquot comprising a staining with an anti-sTn antibody to detect B cells and/or follicular dendritic cells having a determinant comprising sTn on their cell surface (e.g., IgG1 murine mAb) including an incubation with a secondary rabbit anti-mouse IgG antibody labeled with Pe; and then a double-staining with anti-CD19 antibody (IgG1 mAb) labeled with Pe-Cy5, and an anti-CD21 antibody (IgG1 mAb) labeled with FITC (e.g., depending on the cell types present in the preparation being analyzed, to detect B cells comprising CD19+CD21+sTn+ cells, FDC comprising CD19−CD21+sTn+ cells, or to detect both CD19+CD21+sTn+ cells and CD19−CD21+sTn+ cells). Another exemplary combination is triple-staining of an aliquot comprising a staining with an anti-sTn antibody (e.g., IgG1 murine mAb) to detect sTn+ T cells, including an incubation with a secondary rabbit anti-mouse IgG antibody labeled with Pe, and then a double-staining with anti-CD19 antibody (IgG1 mAb) labeled with Pe-Cy5, and an anti-CD5 antibody (IgG1 mAb) labeled with FITC (e.g., to detect CD19-CD5+sTn+ cells).

Any one of several available staining protocols may be used for cell staining. For example, for the first aliquot of cells which remain unstained, the cells were mixed with staining buffer alone (e.g., 50 μl of a physiologically acceptable buffer). The staining buffer utilized was phosphate buffered saline containing 2% fetal calf serum and 0.1% sodium azide. In general, and for contact and mixing an aliquot of cells with one or more affinity ligands, the cells (e.g., sample volume ranging from 20 μl to 100 μl) are incubated with the one or more different, pre-titered affinity ligands for 20-40 minutes at 4° C. After this incubation, the cells in the reaction mixture may be washed in physiologically acceptable buffer, and then may be diluted to a final volume for analysis on the flow cytometer. In continuing with this illustrative embodiment, the second aliquot of cells (as the negative control for the staining process) was mixed with staining buffer and with 1:10 dilutions of an isotype IgG1 labeled with FITC, an isotype IgG1 labeled with Pe, and an isotype IgG1 labeled with Pe-Cy5; and then incubated for 30 minutes in the dark at 4° C. The mixture was then centrifuged at 1500 rpm for 5 minutes. The supernatant was removed and a wash solution (e.g., 150 μl of a physiologically acceptable solution) was used to suspend the cell pellet, and then the mixture was centrifuged (a wash step). The wash step may be repeated one or more times. The cell pellet from the final wash is then taken up in a physiologically acceptable solution in a sufficient volume for flow cytometric analysis (e.g., 200-250 μl). The third aliquot of cells was double-stained using essentially the same protocol as for the second aliquot, except that the antibodies mixed with the cells of the third aliquot were the one or more affinity ligands for determining an amount of mononuclear cell phenotype desired to be determined (e.g., anti-CD19 IgG1 mAb labeled with Pe-Cy5, and an anti-CD21 IgG1 mAb labeled with FITC; final dilution of each mAb was 1:10). In some cases, a fourth aliquot of cells was triple-stained as described above. For example, cells were first mixed and incubated with anti-sTn antibody (murine IgG1), and then washed; followed by mixing and incubating with a secondary rabbit anti-mouse IgG antibody labeled with Pe, and then washed; followed by a double-staining with anti-CD19 antibody labeled with Pe-Cy5, and an anti-CD21 antibody labeled with FITC, and then washed. A number of commercially available flow cytometers can be used as the instrument on which is performed the method of the present invention. Desirably, the flow cytometer has a single laser source; and in a preferred embodiment, the single laser source is an argon laser tuned at 488 nanometers (nm). Additionally, the flow cytometer is operatively connected to appropriate operating software and data management systems.

According to the method of the present invention, determined was an amount of mononuclear cell phenotype in clinical samples obtained from individuals having solid, non-lymphoid tumor and a pro-tumor immune response. Also determined was an amount of mononuclear cell phenotype in clinical samples obtained from apparently healthy individuals, from which determination may be established a reference value. In one illustration of this method, the clinical samples comprised peripheral blood obtained by venipuncture into blood collection tubes, wherein peripheral blood mononuclear cells were isolated and then analyzed; and the respective amounts were determined using flow cytometric methods by the techniques disclosed herein. For example, where mononuclear cell phenotype comprised one or more lymphocyte subpopulations, light scatter was used as a parameter to gate on primarily lymphocytes based on the size, granularity and cell volume of lymphocytes. In addition to gating for light scatter, each sample undergoing the staining process was gated for respective fluorescence emission(s). In continuing with this example, when an amount of memory B cells was determined by double-staining (e.g., for CD19 and CD21), the analysis was gated on those cells positive for CD19 expression as determined by detection of Pe-Cy5 fluorescent emission. In this analysis, CD19 positive lymphocytes were considered to represent the relative overall subpopulation of B cells in the clinical sample analyzed, and were expressed as a percentage of the number of white blood cells in the sample. CD19 positive lymphocytes were then gated for those cells also positive for CD21 expression as determined by detection of FITC fluorescent emission. Lymphocytes double stained for both CD19 and CD21 were considered to represent memory B cells. Such CD19+CD21+ B cells were then expressed in an amount as a percentage of overall B cells by using the formula:
(the number of CD19+CD21+ B cells/number of CD19+ B cells)×100.
A similar procedure was used to determine amounts of B cell subpopulations comprising CD19+CD21++ cells; CD19+sTn+ cells; and CD19+CD21+sTn+ cells (e.g., by triple staining).

In an illustration in which mononuclear cell phenotype comprised sTn+ T cells, after using light scatter to gate on primarily lymphocytes, an amount of overall T cells was determined using double-staining (e.g., CD19− and CD5+), wherein the analysis was gated on those cells positive for CD5 expression as determined by detection of FITC fluorescent emission. CD5 positive cells were then gated for those cells negative for CD19 expression as determined by the absence of detection of Pe-Cy5 fluorescent emission. Lymphocytes stained for CD5, but unstained for CD19, were considered to represent an overall subpopulation of T cells, and were expressed as a percentage of white blood cells in the sample. A similar procedure was used to determine an amount of CD19−CD5+sTn+ T cells by triple staining with anti-sTn antibody with a secondary antibody labeled with Pe, anti-CD19 antibody labeled with Pe-Cy5, and anti-CD5 antibody labeled with FITC. Thus, an amount of CD19−CD5+sTn+ cells was then expressed as a percentage of an overall T cell subpopulation (CD19−CD5+) by using the formula:
(the number of CD19−CD5+sTn+ cells/number of CD19−CD5+ cells)×100.

In an illustration in which mononuclear cell phenotype comprised one or more follicular dendritic cell subpopulations, an amount of FDC was determined using double-staining (e.g., CD19− and CD21+), wherein the analysis was gated on those cells positive for CD21 expression as determined by detection of FITC fluorescent emission. CD21 positive nucleated cells were then gated for those cells negative for CD19 expression as determined by the absence of detection of Pe-Cy5 fluorescent emission. Nucleated cells stained for CD21, but unstained for CD19, were considered to represent FDC. An amount of CD19−CD21+ FDC was then expressed as a percentage of total nucleated cells (“nucleated events”) by using the formula:
(the number of CD19−CD21+ FDC/number of nucleated events)×100.
A similar procedure was used to determine an amount of CD19−CD21+sTn+ FDC by triple staining with anti-sTn antibody with a secondary antibody labeled with Pe, anti-CD19 antibody labeled with Pe-Cy5, and anti-CD21 antibody labeled with FITC. Thus, an amount of CD19−CD21+sTn+ FDC was then expressed as a percentage of total FDC (CD19CD21+) by using the formula:
(the number of CD19−CD21+sTn+ FDC/number of total FDC)×100.

In an illustration of the method according to the present invention wherein lymphoid tissue was used as the clinical sample from which is determined an amount of mononuclear cell phenotype, lymphoid tissue samples were processed by cutting the tissue into thin sections, and performing an enzyme digestion (with collagenase, hyaluronidase, and DNase) to obtain a cell preparation. The cell preparation was then enriched for mononuclear cells using a process as described in Example 1 herein (e.g., density gradient centrifugation); and then stained and analyzed by flow cytometry using essentially the same methods and reagents as described herein for clinical samples comprising peripheral blood.

As previously disclosed herein, mononuclear cell phenotype may comprise one or more mononuclear cell subpopulations. Using the formulas and methods described herein, illustrated in Table 2 are amounts of respective mononuclear cells subpopulations (“MNC”), that may be determined using the method of the present invention. The mononuclear cell subpopulations were determined in clinical samples comprising peripheral blood (“PBL”) or lymphoid tissue (“LT”) from individuals having a pro-tumor immune response and solid, non-lymphoid tumor (“Tumor/PTIR”), and from healthy individuals lacking a pro-tumor immune response (“Reference value”). The amounts were expressed as the Mean percentage±standard error of the mean. Thus, in a preferred embodiment, and as previously described herein in more detail, mononuclear cell phenotype may comprise one or more mononuclear cell subpopulations illustrated in Table 2.

TABLE 2

| | Reference | Tumor/ | Reference | Tumor/ |
|---|---|---|---|---|
| MNC | value PBL | PTIR PBL | value LT | PTIR LT |
| CD19+ | 12.7 ± 3.6 | 2.7 ± 0.5 | — | — |
| CD19+ sTn+ | 1.3 ± 0.4 | 5.9 ± 1.5 | 9.1 ± 4.1 | 36.8 ± 4.8 |
| CD19+ CD21+ | 22.0 ± 3.6 | 58.2 ± 5.2 | 10.2 ± 4.4 | 69.8 ± 6.1 |
| CD19+ CD21++ | 3.8 ± 1.8 | 48.8 ± 5.3 | 1.1 ± 0.1 | 48.8 ± 5.3 |
| CD19+CD21+sTn+ | 29.7 ± 5.0 | 16.1 ± 3.7 | — | — |
| CD19+ CD5+ | 1.8 ± 0.9 | 2.4 ± 1.1 | — | — |
| CD19+CD5+ sTn+ | 0.3 ± 0.1 | 29.0 ± 6.3 | — | — |
| CD19− CD5+ | 52.7 ± 6.6 | 48.5 ± 5.5 | — | — |
| CD19− CD5+sTn+ | 2.1 ± 0.4 | 30.2 ± 7.1 | — | — |
| CD19− CD21+ | 1.0 ± 0.1 | 19.3 ± 3.2 | 1.3 ± 0.9 | 15.9 ± 4.1 |
| CD19−CD21+sTn+ | 0 ± 0.008 | 19.9 ± 4.8 | see FIG. 2 | see FIG. 2 |

Table 2 shows that there are a number of B cell subpopulations which significantly differ in amount in a body fluid comprising peripheral blood of individuals having a pro-tumor immune response as compared to the respective reference value. For example, there is a statistically significant (P value=0.0001) decrease in an amount of overall B cells (e.g., CD19+ cells) in a body fluid comprising peripheral blood of individuals having solid, non-lymphoid tumor and a pro-tumor immune response as compared to the reference value. Additionally, there is a statistically significant (P value<0.0001) increase in an amount of memory B cells (e.g., CD19+CD21+ cells) in a body fluid comprising peripheral blood of individuals having a pro-tumor immune response as compared to the reference value. There is also a statistically significant (P value<0.0001) increase in the relative percentage of memory B cells hyper-expressing CD21 (e.g., CD19+CD21++ cells) in peripheral blood of individuals having a pro-tumor immune response as compared to the reference value. As shown in Table 2, the Mean % ±SEM for peripheral blood sTn+ memory B cells (e.g., CD19+CD21+sTn+ cells) from individuals having solid, non-lymphoid tumor and a pro-tumor immune response is 16.12±3.72; whereas the Mean % ±SEM for the reference value is 29.67±5.0. Using 15% as a threshold value of sTn+ memory cells, 75% of the individuals having solid, non-lymphoid tumor and a pro-tumor immune response have a percentage lower than the threshold value. Thus, there is a statistically significant (P value=0.035) decrease in an amount of peripheral blood sTn+ memory B cells in individuals having a pro-tumor immune response as compared to the values in individuals who lack a pro-tumor immune response. There is also a statistically significant (P value<0.0001) increase in an amount of sTn+ B cells (e.g., CD19+sTn+ cells) in the peripheral blood of individuals having a pro-tumor immune response as compared to the reference value. Additionally, there is also a statistically significant (P value<0.001) increase in an amount of sTn+ B1 cells (e.g., CD19+CD5+sTn+ cells) in the peripheral blood of individuals having a pro-tumor immune response as compared to the reference value.

Table 2 shows that there are a number of B cell subpopulations which significantly differ in amount in a sample comprising lymphoid tissue of individuals having a pro-tumor immune response as compared to the respective reference value. For example, there is a statistically significant (P value=0.001) increase in an amount of memory B cells (e.g., CD19+CD21+ cells) in lymphoid tissues of individuals having a pro-tumor immune response as compared to the reference value. By using a threshold value of 20%, only individuals having a pro-tumor immune response have a percentage of lymphoid tissue CD19+CD21+ B cells greater than the threshold value. The amount of lymphoid tissue memory B cells could possibly be even greater if only examined was lymphoid tissue which is a foci of a pro-tumor immune response (as opposed to analysis of lymphoid tissue that was picked at random). There is also a statistically significant (P value<0.02) increase in an amount of memory B cells hyperexpressing CD21 (e.g., CD19+CD21++ cells) in lymphoid tissues in individuals having a pro-tumor immune response as compared to the reference value. Also, there is a statistically significant (P value<0.03) increase in an amount of sTn+ B cells (e.g., CD19+sTn+ cells) in lymphoid tissue in individuals having a pro-tumor immune response as compared to the reference value.

Table 2 shows that there are a number of follicular dendritic cell subpopulations which significantly differ in amount in a body fluid comprising peripheral blood of individuals having a pro-tumor immune response as compared to the respective reference value. For example, in a body fluid comprising peripheral blood, there is a statistically significant (P value<0.01) increase in an amount of sTn+ FDC (e.g., CD19−CD21+sTn+ cells) in individuals having a pro-tumor immune response as compared to the reference value; and a statistically significant (P value<0.01) increase in an amount of overall FDC (e.g., CD19−CD21+ cells) in individuals having a pro-tumor immune response as compared to the reference value. Table 2 also shows that there are a number of follicular dendritic cell subpopulations which significantly differ in amount in lymphoid tissue of individuals having a pro-tumor immune response as compared to the respective reference value. For example, there is a statistically significant (P value=0.005) increase in an amount of an overall FDC population (e.g., CD19−CD21+ cells) in individuals having a pro-tumor immune response as compared to the reference value. Using a threshold value of 5% of lymphoid tissue FDC (e.g., CD19−CD21+ cells), only individuals having a pro-tumor immune response have a percentage of lymphoid tissue FDC greater than the threshold value in all samples tested to date (thus, such a threshold value is above all values obtained from healthy controls tested). The amount of lymphoid tissue FDC could possibly be even greater if only examined was lymphoid tissue which is a foci of a pro-tumor immune response (as opposed to analysis of lymphoid tissue that was picked at random).

Using the methods outlined above, an amount of sTn+ FDC (e.g., CD19−CD21+sTn+ cells) from lymphoid tissue of individuals having a pro-tumor immune response was compared to a reference value. As shown in FIG. 2, such a determination identifies two distinct subpopulations of sTn+ FDC from lymphoid tissue of individuals having solid, non-lymphoid tumor and a pro-tumor immune response (“Cancer”). As shown by FIG. 2, by using a threshold value consisting essentially of a range comprising the reference value from a low of about 35% (threshold value_{LOW}) to a high of about 50% (threshold value_{HIGH}), 25% of individuals having a pro-tumor immune response have an amount of lymphoid tissue sTn+ FDC greater than threshold value_{HIGH}. While not intending to be bound by theory, these individuals have lymphoid tissues that appear to be highly active foci for a pro-tumor immune response, as evidenced by a predominance of sTn positivity by the FDC. 75% of individuals having a pro-tumor immune response have a percentage of lymphoid tissue CD19−CD21+sTn+ FDC lower than the threshold value_{LOW}. While not intending to be bound by theory, these individuals have lymphoid tissues that appear to represent foci of a pro-tumor immune response against at least one shed tumor antigen other than that containing an epitope comprising terminal sTn (e.g., a terminal carbohydrate epitope other than sTn, such as an epitope comprising a terminal sialic acid 2,6-linked to galactose).

Table 2 shows that there are a number of T cell subpopulations which significantly differ in amount in a body fluid comprising peripheral blood of individuals having a pro-tumor immune response as compared to the respective reference value. For example, there is a statistically significant (P value<0.001) increase in an amount of sTn+ T cells (e.g., CD19−CD5+sTn+ cells) in peripheral blood of individuals having a pro-tumor immune response as compared to the reference value.

### EXAMPLE 3

This Example further illustrates embodiments of the method according to the present intention for determining an amount of mononuclear cell phenotype in a clinical sample from an individual as an indicator (a diagnostic value, or a prognostic value) related to a pro-tumor immune response. As illustrated in Table 2, an indicator may comprise alterations in amount of mononuclear cell phenotype as determined from assaying clinical samples from individuals having a pro-tumor immune response. As apparent to one skilled in the art from the descriptions herein, such an indicator may be expressed in several ways. For illustrative examples, consider Formula_{I} and Formula_{II}.
(% tB<T_{B}) and [(% mB_{CD19+cD21+}>T_{19+CD21+}) or (% mB_{CD19+CD21++}>T_{19+CD21++}) or (% B_{CD19+sTn+}>T_{19+sTn+}) or (% mB_{CD19+CD21+sTn+}<T_{19+CD21+sTn+}) or (% B_{CD19+CD5+sTn+}>T_{19+CD5+sTn+}), or (% T_{CD19-CD5+sTn+}>T_{19-CD5+sTn+}) or a combination thereof]=Cancer and PTIR Formula_{I}
(% mB_{CD19+CD21+}>T_{19+CD21+}) or (% mB_{CD19+CD21++}T_{19+CD21++}) or (% B_{CD19+sTn+}>T_{19+sTn+}) or (% mB_{CD19+CD21+sTn+}<T_{19+CD21+sTn+}) or (% B_{CD19+CD5+sTn+}>T_{19+CD5+sTn+}), or (% T_{CD19-CD5+sTn+}>T_{19-CD5+sTn+}) or a combination thereof=PTIR Formula_{II}
In the above-representative formulas, % tB is an amount of overall number of B cells (e.g., % of CD19+ B cells) determined from assaying a clinical sample comprising peripheral blood from an individual being screened for a pro-tumor immune response; T_{B} is equal to a threshold value comprising the minimum amount of overall B cells determined from assaying peripheral blood of healthy individuals (e.g., Mean−2.5(SEM); or 3.7%); % mB_{CD19+CD21+} is an amount (e.g., %) of memory B cells (e.g., CD19+CD21+ cells) determined from assaying a clinical sample comprising peripheral blood from an individual being screened for a pro-tumor immune response; T_{19+CD21+} is equal to a threshold value comprising the maximum amount of memory B cells (e.g., CD19+CD21+ cells determined from assaying peripheral blood of healthy individuals (e.g., Mean+2.5(SEM); or 31%); % mB_{CD19+CD21++} is an amount (e.g., %) of CD21 hyperexpressing memory B cells (e.g., CD19+CD21++ cells) determined from assaying a clinical sample comprising peripheral blood from an individual being screened for a pro-tumor immune response; T_{19+CD21++} is equal to a threshold value comprising a maximum amount of memory B cells (e.g., CD19+CD21++ cells) determined from assaying peripheral blood of healthy individuals (e.g., Mean+2.5(SEM); or 8.3%); % B_{CD19+sTn+} is an amount (e.g., %) of sTn+ B cells (e.g., CD19+sTn+ cells) determined from assaying a clinical sample comprising peripheral blood from an individual being screened for a pro-tumor immune response; T_{19+sTn+} is equal to a threshold value comprising a maximum amount of sTn+ B cells (e.g., CD19+sTn+ cells) determined from assaying peripheral blood of healthy individuals (e.g., Mean+2.5(SEM); or 2.3%); % mB_{CD19+cD21+sTn+} is an amount (e.g., %) of sTn+ memory B cells (e.g., CD19+CD21+sTn+ cells) determined from assaying a clinical sample comprising peripheral blood from an individual being screened for a pro-tumor immune response; T_{19+CD21+sTn+} is equal to a threshold value comprising a minimum amount of sTn+ memory B cells (e.g., CD19+CD21+sTn+ cells) determined from assaying peripheral blood of healthy individuals (e.g., Mean−2.5(SEM); or 17.4%); % B_{CD19+CD5+sTn+} is an amount (e.g., %) of sTn+ B1 cells (e.g., CD19+CD5+sTn+ cells) determined from assaying a clinical sample comprising peripheral blood from an individual being screened for a pro-tumor immune response; T_{19+CD5+sTn+} is equal to a threshold value comprising a maximum amount of sTn+ B1 cells (e.g., CD19+CD5+sTn+ cells) determined from assaying peripheral blood of healthy individuals (e.g., Mean+2.5(SEM); or 0.6%); % T_{CD19-CD5+sTn+} is an amount (e.g., %) of sTn+ T cells (e.g., CD19−CD5+sTn+ cells) determined from assaying a clinical sample comprising peripheral blood from an individual being screened for a pro-tumor immune response; T_{19-cD5+sTn+} is equal to a threshold value comprising a maximum amount of sTn+ T cells (e.g., CD19−CD5+sTn+ cells) determined from assaying peripheral blood of healthy individuals (e.g., Mean+2.5(SEM); or 3.1%); PTIR is an abbreviation for the presence of a pro-tumor immune response; and “Cancer” is indicative of the presence of a solid, non-lymphoid tumor. Thus, using Formula_{I}, a difference in an amount of overall B cells (as compared to a reference value for overall B cells) combined with a difference in any one or more of B cell subpopulations (as compared to a respective reference value) comprising memory B cells, sTn+ memory B cells, sTn+ B cells, sTn+ B1 cells, CD21 hyperexpressing memory B cells, and sTn+ T cells, translates into a 99.8% probability that the individual has a pro-tumor immune response and a solid, non-lymphoid tumor. For example, where the individual's peripheral blood has a % of CD19+ B cells less than 3.7%, and where the individual's peripheral blood has an amount of a mononuclear cell subpopulation comprising one or more of: CD19+CD21+ cells of greater than 31%, CD19+CD21++ cells of greater than 8.3%, CD19+sTn+ cells of greater than 2.3%, CD19+CD5+sTn+ cells of greater than 0.6%, CD19+CD21+sTn+ cells less than 17.4%, and sTn+ T cells of greater than 3.1%, then there is a 99.8% probability that the individual has both a pro-tumor immune response and a solid non-lymphoid tumor. Generally then, Formula_{I} illustrates the generation of indicators to identify individuals as having or lacking a pathological condition, in providing an additional parameter to a competent health professional in making a medical opinion. Similarly, using the Formula_{II}, a significant difference in an amount of mononuclear cell phenotype (as compared to a reference value) comprising one or more mononuclear cell subpopulations designated therein may result in a 99.8% probability that the individual has a pro-tumor immune response. For example, where the individual's peripheral blood has a mononuclear cell subpopulation comprising one or more of: CD19+CD21+ cells of greater than 31%, CD19+CD21++ cells of greater than 8.3%, CD19+sTn+ cells of greater than 2.3%, CD19+CD5+sTn+ cells of greater than 0.6%, CD19+CD21+sTn+ B cells less than 17.4%, and sTn+ T cells of greater than 3.1%, then there is a 99.8% probability that the individual has a pro-tumor immune response. Generally then, Formula_{II} illustrates the generation of indicators to identify individuals as having or lacking a pathological condition, in providing an additional parameter to a competent health professional in making a medical opinion.

Similarly, and with regard to an indicator related to FDC for purposes of illustration but not limitation, consider Formula_{III}.
(% FDC_{CD19-CD21+STn+}>T_{CD19-CD21+STn+}) or (% FDC_{CD19-CD21+}<T_{CD19-CD21+}) P-TIR or P-TIR & Cancer
wherein % FDC_{CD19-CD21+sTn+} is an amount (e.g., %) of sTn+ FDC (e.g., CD19−CD21+sTn+ cells) determined from assaying a clinical sample comprising peripheral blood from an individual being screened for a pro-tumor immune response; T_{CD19-CD21+} is equal to a threshold value comprising a maximum amount of sTn+ FDC (e.g, CD19−CD21+sTn+ cells) determined from assaying peripheral blood of healthy individuals (e.g., Mean−2.5(SEM); or less than 1%); % FDC_{CD19-CD21+} is an amount (e.g., %) of FDC (e.g., CD19−CD21+ cells) determined from assaying a clinical sample comprising lymphoid tissue from an individual being screened for a pro-tumor immune response; and T_{CD19-CD21+} is equal to a threshold value comprising a maximum amount of FDC (e.g., CD19−CD21+ cells) determined from assaying lymphoid tissue of healthy individuals (e.g., Mean+2.5(SEM); or about 4%); or % FDC_{CD19-CD21+} is an amount (e.g., %) of FDC (e.g., CD19−CD21+ cells) determined from assaying a clinical sample comprising peripheral blood from an individual being screened for a pro-tumor immune response; and T_{CD19-CD21+} is equal to a threshold value comprising a maximum amount of FDC (e.g., CD19−CD21+ cells) determined from assaying peripheral blood of healthy individuals (e.g., Mean+2.5(SEM); or about 1.3%). Thus, using the illustrated Formula_{III}, a significant difference in a mononuclear cell phenotype (as compared to a reference value), comprising the one or more mononuclear cell subpopulations, may translate into a greater than 95% probability that the individual has a pro-tumor immune response, or a pro-tumor immune response and solid, non-lymphoid tumor. For example, and as compared to the control values, where the individual's peripheral blood has a % of CD19−CD21+sTn+ FDC greater than about 1%, and/or where the individual's lymphoid tissue has a % of CD19−CD21+ FDC greater than 4%, and/or where an individual's peripheral blood has a % of CD19−CD21+ FDC greater than 1.3%, then there is a greater than 99% probability that the individual has either a pro-tumor immune response or both a pro-tumor immune response and a solid non-lymphoid tumor. It will be apparent to one skilled in the art that a mononuclear cell phenotype may combine mononuclear cell subpopulations indicated in Formula_{III} with any one or more of those indicated Formula_{I} and/or Formula_{II}.

### EXAMPLE 4

This Example further illustrates an embodiment of the method according to the present invention for determining an amount of mononuclear cell phenotype by assaying a clinical sample from an individual in generating an indicator (a diagnostic value, or a prognostic value) related to a pro-tumor immune response. Previously described herein in more detail is the use of a prognostic indicator in monitoring anticancer therapy and effects, if any, of the anticancer therapy on any one or more of tumor and a pro-tumor immune response. Essentially, an amount of mononuclear cell phenotype determined from analysis of a reference sample (e.g., obtained prior to the initiation or before the conclusion of anticancer therapy) from an individual, is compared to an amount of mononuclear cell phenotype determined from assaying a test sample obtained during anticancer therapy but after the time at which the reference sample was obtained, or obtained post anticancer therapy. The comparison is made to determine whether there is a difference in amount of mononuclear cell phenotype determined from assaying the test sample as compared to the amount of mononuclear cell phenotype determined from assaying the reference sample. As an illustration of this embodiment, an individual having a localized colon tumor and a pro-tumor immune response underwent anticancer therapy comprising surgical resection of the tumor followed by multiple regimens of chemotherapeutic treatment. FIG. 3 illustrates a mononuclear cell phenotype comprising an amount of overall B cells (e.g., CD19+ cells), an amount of CD21 hyper-expressing memory B cells (e.g., CD19+CD21++ cells), and an amount of sTn+ B cells (e.g., CD19+sTn+ cells), after surgery but before chemotherapy (□) and after chemotherapy (▪), and as compared to a reference value established from apparently healthy individuals (]N). Anticancer therapy comprising surgical removal of the tumor resulted in an increase in the amount of overall B cells to within a range observed in the reference value (e.g., 8.7%). As shown in FIG. 3, as a result of three treatments with chemotherapy, the amount of overall B cells remain within the reference values (27%, and after correction for the leukopenia, about 12%). The chemotherapy significantly reduced the amount of CD19+CD21++ B cells from 14.6% to within a range observed in the reference value (e.g., 1.1%). Likewise, chemotherapy significantly reduced the amount of sTn+ B cells (e.g., CD19+sTn+ cells) from 14.6% to a level approaching the range of the reference value (e.g., 3.2%). Such an observed effect of anticancer therapy on alteration of mononuclear cell phenotype associated with tumor and a pro-tumor immune response may be an indicator that the combined anticancer therapy (surgery and chemotherapy) has effected a substantial reduction of tumor burden and is reducing the pro-tumor immune response in the treated individual.

As another illustration of this embodiment, an individual having Stage 1V colon cancer with liver metastases and a pro-tumor immune response underwent anticancer therapy comprising surgical resection of the tumor and metastases followed by multiple regimens of chemotherapy. FIG. 4 illustrates a mononuclear cell phenotype comprising an amount of overall B cells (e.g., CD19+ cells), memory B cells (e.g., as CD19+CD21+ cells), and sTn+ B cells (e.g., CD19+sTn+ cells) approximately 1 year after anticancer therapy (▪). As shown in FIG. 4, 1 year after anticancer therapy, the amount of overall B cells is below a range observed for the reference value (e.g., 2.9%), an indicator suggestive of residual or recurrent tumor. The amount of memory B cells is a value within a range observed for the reference value (e.g., 2.0%). However, the amount of sTn+ B cells is significantly increased to a level observed in individuals having a pro-tumor immune response (e.g., 46.3%). From this determination, the combined mononuclear cell subpopulations comprise a mononuclear cell phenotype which may comprise an indicator that the individual (a) has a high probability for recurrence and/or has recurrence of tumor, or has residual tumor; and (b) has a pro-tumor immune response. Generally, such indicators provide an additional parameter to a competent health professional in making a medical decision concerning the efficacy of, or need for additional, anticancer therapy.

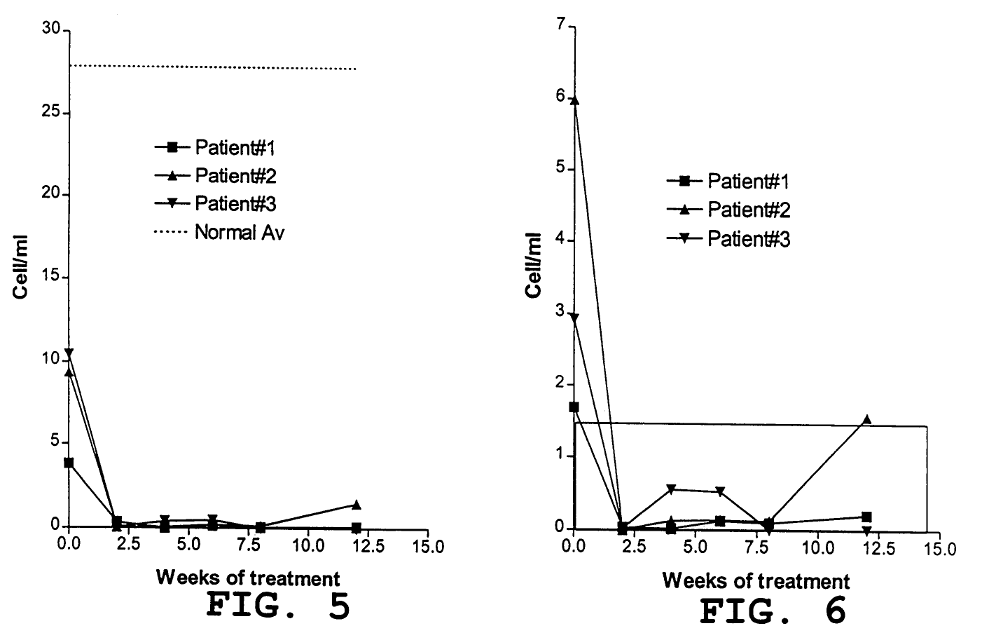

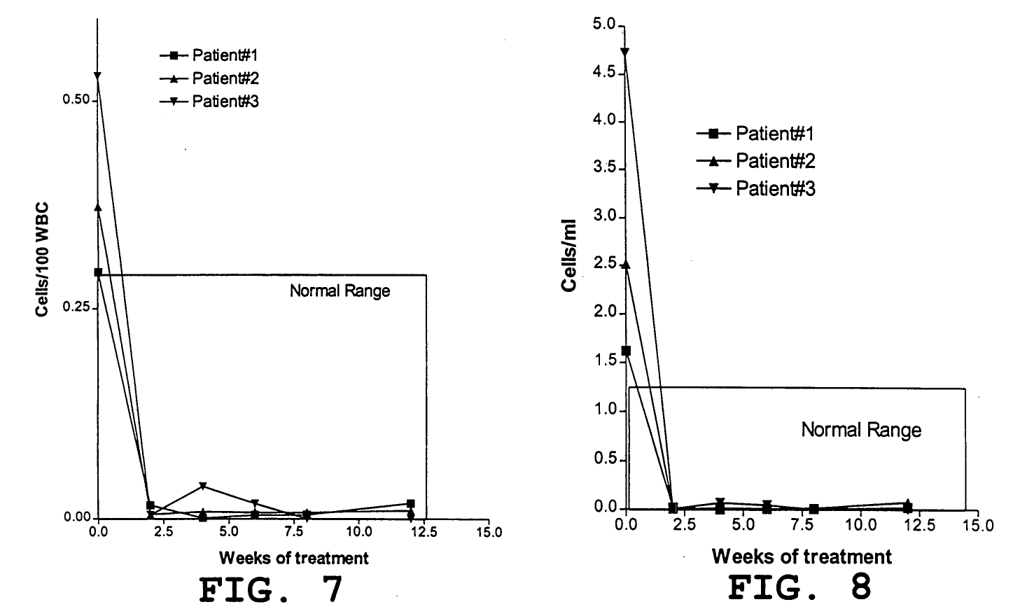

To illustrate another embodiment in which the amount of mononuclear cell phenotype is used as an indicator to monitor efficacy of anticancer therapy, three individuals having a pro-tumor immune response were treated with an immunotherapeutic composition for depleting B cells. The regimen of treatment comprised three administrations of the composition: the initial treatment (week 0), one at week 4, and one at week 8. A reference sample (day 0) was obtained, and test samples were obtained during and after the treatment period, from which peripheral blood samples was determined mononuclear cell phenotype comprising overall B cells (e.g., CD19+ cells), sTn+ B cells (e.g., CD19+sTn+ cells), memory B cells (e.g., CD19+CD21+ cells), and sTn+ memory B cells (e.g., CD19+CD21+sTn+ cells). As shown in FIGS. 5-8, the anticancer therapy of a pro-tumor immune response (as directed against B cells) of the 3 individuals (▪▴,▾) resulted in a depletion in an amount of overall B cells (FIG. 5), and normalization (e.g., to within the normal range of clinical values as indicated by the boxed area) of the amounts of sTn+ B cells (FIG. 6), sTn+ cells (FIG. 7), and memory B cells (FIG. 8). Thus, that the alteration in mononuclear cell phenotype observed before treatment was corrected to within a reference value after initiation of treatment, as illustrated in FIGS. 6-8, is an indicator that the anticancer therapy was effective in treating a pro-tumor immune response in the treated individuals.

### EXAMPLE 5

This Example illustrates embodiments of assay kits according to the present invention for performing methods for determining an amount of mononuclear cell phenotype comprising one or more mononuclear cell subpopulations in a clinical sample from an individual. As apparent to those skilled in the art, the assay kits may include various components, depending on the complexity of the screening method utilized for determining an amount of at least one mononuclear cell subpopulation comprising mononuclear cell phenotype. An assay kit contains affinity ligands that facilitate determination of an amount of mononuclear cell phenotype that may be present in the sample analyzed. In a preferred embodiment, the affinity ligands included in the kit according to the present invention comprise one or more first affinity ligands having binding specificity for a determinant selected from the group consisting of a pan B cell marker, a pan T cell marker, a pan FDC marker, and a combination thereof; and one or more second affinity ligands having binding specificity for a determinant comprising a marker expressed by mononuclear cells as a response to an immune process (as previously described herein in more detail). It will be apparent that the one or more first affinity ligands may be used in combination with the one or more second affinity ligands in assaying a sample for determination of an amount of mononuclear cell phenotype. The kit may further comprise one or more additional affinity ligands that may be used to determine one or more additional mononuclear cell subpopulations comprising mononuclear cell phenotype.

In one preferred embodiment, the kit comprises one or more first affinity ligands having binding specificity for a determinant selected from the group consisting of a pan B cell marker, a pan T cell marker, and a combination thereof; and a second affinity ligand for detecting sTn. For example, in one preferred assay kit, the kit comprises a first affinity ligand for binding and detecting a pan B cell marker comprising CD19, and a second affinity ligand for binding and detecting sTn; e.g., for determining a mononuclear cell phenotype comprising the amount of overall B cells (e.g., CD19+ cells) and sTn+ B cells (e.g., CD19+sTn+ cells). In another example, a preferred kit comprises a first affinity ligand for binding and detecting a pan T cell cell marker comprising CD3, and a second affinity ligand for binding and detecting sTn; e.g., for determining an amount of mononuclear cell phenotype comprising the amount of overall T cells (e.g., CD3+ cells) and sTn+ T cells (e.g., CD3+ sTn+ cells). In another example, a preferred kit comprises a plurality of affinity ligands, wherein a first affinity ligand is for binding and detecting a determinant comprising a pan B cell marker (e.g., CD19), a second affinity ligand is for binding and detecting a determinant comprising a pan T cell marker (e.g., CD3), and a third affinity ligand is for binding and detecting sTn. Thus, the kit may be used to determine a mononuclear cell phenotype comprised of an amount of overall B cells (e.g., CD19+ cells), an amount of sTn+ B cells (e.g., CD19+sTn+ cells), an amount of overall T cells (e.g., CD3+ cells), and an amount of sTn+ T cells (e.g., CD3+sTn+ cells). In another illustrative embodiment, a preferred kit comprises a plurality of affinity ligands, wherein a first affinity ligand is for binding and detecting CD19, a second affinity ligand is for binding and detecting CD5, and a third affinity ligand is for binding and detecting sTn. Thus, the kit may be used to determine a mononuclear cell phenotype comprising an amount of overall B cells (e.g., CD19+ cells), an amount of sTn+ B cells (e.g., CD19+sTn+ cells), an amount of B1 cells (e.g., CD19+CD5+ cells), an amount of sTn+ B1 cells (e.g., CD19+CD5+sTn+ cells), an amount of overall T cells (e.g., CD5+ CD19cells), and an amount of sTn+ T cells (e.g., CD5+ CD19−sTn+ cells); or other combinations thereof.

In another preferred embodiment, the kit comprises a plurality of affinity ligands comprising one or more first affinity ligands for binding and detecting for detecting a determinant comprising a pan lymphocyte marker, one or more second affinity ligands for binding and detecting a determinant comprising a marker expressed by mononuclear cells as a response to an immune process (as previously described herein in more detail), and one or more third affinity ligands for binding and detecting a determinant comprising a pan follicular dendritic cell marker. For example, a preferred kit may comprise a plurality of affinity ligands, wherein a first affinity ligand is for binding and detecting CD19, a second affinity ligand is for binding and detecting a CD21, and a third affinity ligand is for binding and detecting sTn. Thus, the kit may be used to determine mononuclear cell phenotype comprising an amount of overall B cells (e.g., CD19+ cells), an amount of sTn+ B cells (e.g., CD19+sTn+ cells), an amount of memory B cells (e.g., CD19+CD21+ cells), an amount of sTn+ memory B cells (e.g., CD5+ CD19−sTn+ cells), an amount of CD21 hyper-expressing memory B cells (e.g., CD19+CD21++ cells), an amount of overall follicular dendritic cells (e.g., CD19−CD21+ cells), and sTn+ follicular dendritic cells (e.g., CD19−CD21+sTn+ cells). In another example, a preferred kit may comprise a plurality of affinity ligands, wherein a first affinity ligand is for binding and detecting CD19, a second affinity ligand is for binding and detecting CD21, a third affinity ligand is for binding and detecting sTn, and a fourth affinity ligand is for binding and detecting CD5. Thus, the kit may be used to determine a mononuclear cell phenotype comprising an amount of overall B cells (e.g., CD19+ cells), an amount of sTn+ B cells (e.g., CD19+sTn+ cells), an amount of B1 cells (e.g., CD19+CD5+ cells), an amount of sTn+ B1 cells (e.g., CD19+CD5+sTn+ cells), an amount of memory B cells (e.g., CD19+CD21+ cells), an amount of sTn+ memory B cells (e.g., CD5+CD19−sTn+ cells), an amount of CD21 hyperexpressing memory B cells (e.g., CD19+CD21++ cells), an amount of overall T cells (e.g., CD5+ CD19cells), an amount of sTn+ T cells (e.g., CD5+ CD19−sTn+ cells), an amount of overall follicular dendritic cells (e.g., CD19−CD21+ cells), and sTn+follicular dendritic cells (e.g., CD19−CD21+sTn+ cells)); or other combinations thereof.

Additionally, an assay kit of each of the above-described embodiments may further comprise an isotype affinity ligand for each antibody type of affinity ligand used (see, e.g., Example 2 herein for more detail) for determining an amount of mononuclear cell phenotype. In another illustrative example, wherein an aptamer is used as the affinity ligand, an aptamer of the same general backbone sequence (e.g., differing primarily only in the sequence conferring binding specificity) may be used as an isotype affinity ligand. The assay kit according to the present invention may further comprise a reagent comprising a known amount of reference mononuclear cells (comprising one or more mononuclear cell subpopulations detectable by the kit) for use in comparatively determining alterations in mononuclear cell phenotype in a clinical sample. More particularly, and as apparent to those skilled in the art, the known amount of reference mononuclear cells may be used as an assay control, one or more assay standards, or a combination thereof. For example, and depending on the choice of affinity ligands included in the assay kit, the control may comprise an amount of one or more mononuclear cell subpopulations selected from the group consisting of overall B cells, sTn+ B cells, memory B cells, sTn+ memory B cells, CD21 hyper-expressing B cells, B1 cells, sTn+ B1 cells, overall T cells, sTn+ T cells, overall follicular dendritic cells, sTn+ follicular dendritic cells. Thus, there may be a separate container of reference mononuclear cells for each of mononuclear cell subpopulation comprising mononuclear cell phenotype according to the present invention; or there may be a container of reference mononuclear cells containing a combination of mononuclear cell subpopulations comprising mononuclear cell phenotype. The reference mononuclear cells may be stored in a solution, or may be lyophilized for reconstitution, frozen, or a combination thereof. The reference mononuclear cells may be fixed by prior treatment with any one of a number of solutions known in the art to include, but are not limited to, 1% paraformaldehyde, methanol, methanol/acetone, acetone, 2% (v/v) paraformaldehyde and acetone, and 70% ethanol. The reference mononuclear cells may further comprise pre-stained cells, e.g., stained with multiple affinity ligands for determining the one or more mononuclear cell subpopulations present. The reference mononuclear cells may comprise a series of standards that may comprise a standard representative of a threshold value characteristic of an alteration in mononuclear cell phenotype, and a standard representative of a reference value characteristic of a normal range of clinical values as established for the mononuclear cell phenotype (e.g., as established from apparently healthy individuals). For purposes of illustration only, and not limitation, a standard comprising a threshold value for an alteration in mononuclear cell phenotype may be derived from the values illustrated in Table 2 herein (Tumor/PTIR with respect to each mononuclear cell subpopulation), whereas a standard comprising a reference value may be derived from the values illustrated in Table 2 herein (Reference interval, with respect to each mononuclear cell subpopulation) (see also Formula_{I}, Formula_{II}, Formula_{III}).

It will be apparent to one skilled in the reference mononuclear cells may comprise one or more cell lines are known in the art, including, but not limited to: B-cell lines expressing CD19, CD21, and CD22; an EBV-positive B cell line (“BEVA”) expressing CD19, CD20, and CD21; an EBV-positive B cell line (“Jijoye-P3HR-1”) strongly expressing CD19 and CD20 with weak expression of CD21; CD5+ T cell lines; an immortalized FDC line expressing Ki-M4 and other surface antigens of human FDC origin (“FDC-H1”); EBV-transformed FDC cell lines; and FDC tumor cell lines expressing CD21, Ki-M4, R4/23, and other human FDC markers; and the like. As will be apparent to those skilled in the art, the amount of reference mononuclear cells comprising a known amount, may vary depending upon such factors which include, but are not limited to, the type of clinical sample analyzed (e.g., origin or tissue type), the nature of the one or more affinity ligands used (binding specificity, detectable moiety, etc.), and the methodology and instrumentation used to detect and quantitate mononuclear cell phenotype present in the sample. Additionally, it will be apparent to one skilled in the art that the methods of the present invention can also be carried out in conjunction with other diagnostic and prognostic tests in providing more information regarding a pathological condition, if detected.

The foregoing description of the specific embodiments of the present invention have been described in detail for purposes of illustration. In view of the descriptions and illustrations, others skilled in the art can, by applying, current knowledge, readily modify and/or adapt the present invention for various applications without departing from the basic concept, and therefore such modifications and/or adaptations are intended to be within the meaning and scope of the appended claims.

1-47. (canceled)

48. A method for screening for a pro-tumor immune response indicative of a non-lymphoid tumor in an individual supplying a clinical sample comprising one or more of, B lymphocyte cells, T lymphocyte cells, and follicular dendritic cells, by assaying the clinical sample from the individual, the method comprising:
contacting the clinical sample with a plurality of, antibodies, immunoreactive fragments, peptides, and aptamers, capable of binding to determinants on at least some cells contained in the clinical sample;
detecting the plurality of, antibodies, immunoreactive fragments, peptides, and aptamers, bound to the determinants on the cells in the clinical sample for measuring one or more subpopulations of the cells for estimating an amount of a mononuclear cell phenotype in the individual;
obtaining a difference between the amount of the mononuclear cell phenotype in the individual and a reference value for the mononuclear cell phenotype; and
identifying that a pro-tumor response is present in the individual based, at least in part, on the difference between the amount of the mononuclear cell phenotype in the individual and the reference value for the mononuclear cell phenotype, where the pro-tumor response promotes one or more of, tumor growth, invasion, and metastasis.

49. The method of claim 48, where measuring the one or more subpopulations includes measuring cells comprising B lymphocyte cells.

50. The method of claim 49, where the B lymphocyte cells comprise one or more of sTn+ B cells, memory B cells, CD21 hyperexpressing memory B cells, sTn+ memory B cells, B1 cells, and sTn+ B1 cells.

51. The method of claim 49, where the B lymphocyte cells comprise one or more of, memory B cells, sTn+ B cells, and B1 cells.

52. The method of claim 51, where the memory B cells comprise one or more of, CD21 hyperexpressing memory B cells, and sTn+ memory B cells.

53. The method of claim 51, where the sTn+ B cells comprise one or more of, sTn+ memory B cells, and sTn+ B1 cells.

54. The method of claim 51, where the B1 cells comprise sTn+ B1 cells.

55. The method of claim 48, where measuring the one or more subpopulations includes measuring cells comprising T lymphocyte cells.

56. The method of claim 55, where the T lymphocyte cells comprise sTn+ T cells.

57. The method of claim 48, where measuring the one or more subpopulations includes measuring cells comprising follicular dendritic cells.

58. The method of claim 57, where the follicular dendritic cells comprise sTn+ follicular dendritic cells.

59. The method of claim 48, where measuring the one or more subpopulations includes measuring cells comprising sTn+ cells.

60. The method of claim 59, where the sTn+ cells comprise one or more of, sTn+ B cells, sTn+ T cells, and sTn+ follicular dendritic cells.

61. The method of claim 60, where the sTn+ B cells comprise one or more of, sTn+ memory B cells, and sTn+ B1 cells.

62. A method for determining the status of a pro-tumor immune response in an individual, comprising:
assaying a clinical sample comprising one of, peripheral blood, body fluids other than peripheral blood, and lymphoid tissue, with two or more affinity ligands capable of binding to determinants on at least some cells in the clinical sample, where a first affinity ligand is capable of binding to a determinant comprising a pan B lymphocyte cell marker, and a second affinity ligand is capable of binding to one or more of, a determinant comprising a memory B cell marker, a determinant comprising a B1 cell marker, and a determinant comprising an epitope including a terminal alpha 2,6-linked sialic acid;
detecting at least some of the two or more affinity ligands bound to the determinants on the cells in the clinical sample and estimating an amount of a mononuclear cell phenotype in the individual based, at least in part, on the amount of one or more of, B lymphocyte cells and subpopulations thereof, T lymphocyte cells and subpopulations thereof, and follicular dendritic cells and subpopulations thereof;
obtaining a difference between the mononuclear cell phenotype in the individual and a reference value, the reference value including an earlier value from the same individual; and
based at least in part on the difference, identifying a pro-tumor immune response associated with a non-lymphoid tumor in the individual

63. The method of claim 62, where the pan B lymphocyte cell marker comprises one or more of, CD19, CD20, and CD72.

64. The method of claim 62, where the first and second affinity ligands comprise one or more of, antibodies, immunoreactive fragments, peptides, and aptamers, and the second affinity ligand is capable of binding to a determinant comprising a memory B cell marker.

65. The method of claim 64, where the memory B cell marker comprises one or more of, CD21, CD75, CD45R, and CD22.

66. The method of claim 64, further comprising assaying the clinical sample with a third affinity ligand comprising one or more of, antibodies, immunoreactive fragments, peptides, and aptamers, where the third affinity ligand is capable of binding to a determinant comprising an epitope including a terminal alpha 2,6-linked sialic acid.

67. The method of claim 66, where the epitope including the terminal alpha 2,6-linked sialic acid comprises sTn.

68. The method of claim 62, where the first and second affinity ligands comprise one or more of, antibodies, immunoreactive fragments, peptides, and aptamers, and the second affinity ligand is capable of binding to a determinant comprising a B1 cell marker.

69. The method of claim 68, where the B1 cell marker comprises CD5.

70. The method of claim 68, further comprising assaying the clinical sample with a third affinity ligand comprising one or more of, antibodies, immunoreactive fragments, peptides, and aptamers, where the third affinity ligand is capable of binding to a determinant comprising an epitope including a terminal alpha 2,6-linked sialic acid.

71. The method of claim 70, where the epitope including the terminal alpha 2,6-linked sialic acid comprises sTn.

72. The method of claim 62, where the first and second affinity ligands comprise one or more of, antibodies, immunoreactive fragments, peptides, and aptamers, and the second affinity ligand is capable of binding to a determinant comprising an epitope including a terminal alpha 2,6-linked sialic acid.

73. The method of claim 72, where the epitope including the terminal alpha 2,6-linked sialic acid comprises sTn.

74. The method of claim 72, further comprising assaying the clinical sample with a third affinity ligand comprising one or more of, antibodies, immunoreactive fragments, peptides, and aptamers, where the third affinity ligand is capable of binding to a determinant comprising a memory B cell marker.

75. The method of claim 74, where the memory B cell marker comprises one or more of, CD21, CD75, CD45R, and CD22.

76. The method of claim 72, further comprising assaying the clinical sample with a third affinity ligand comprising one or more of, antibodies, immunoreactive fragments, peptides, and aptamers, where the third affinity ligand is capable of binding to a determinant comprising a B1 cell marker.

77. The method of claim 76, where the B1 cell marker comprises CD5.

78. A method for screening an individual for a pro-tumor immune response by assaying a clinical sample obtained from the individual, the method comprising:
(a) contacting the clinical sample with a plurality of affinity ligands for detecting a mononuclear cell phenotype,
(i) wherein the plurality of affinity ligands comprises an affinity ligand having binding specificity for CD19, an affinity ligand having binding specificity for sTn, and an affinity ligand having binding specificity for CD21, and
(ii) wherein a mononuclear cell phenotype is selected from the group consisting of CD19+sTn+ cells, CD19+CD21+sTn+ cells, CD19+CD21+ cells, CD19+CD21++ cells, CD19−CD21+sTn+ cells, CD19−CD21+ cells, and a combination thereof;
(b) comparing the amount of mononuclear cell phenotype determined from the clinical sample to a reference value for the mononuclear cell phenotype; and
(c) wherein a difference in the amount of mononuclear cell phenotype determined from the clinical sample as compared to the reference value for the mononuclear cell phenotype comprises an indicator of the pro-tumor immune response.

79. The method according to claim 78, wherein the plurality of affinity ligands further comprises an affinity ligand having binding specificity for CD5, and wherein a mononuclear cell phenotype is selected from the group consisting of CD19+sTn+ cells, CD19+CD21+sTn+ cells, CD19+CD21+ cells, CD19+CD21++ cells, CD19−CD21+sTn+ cells, CD19−CD5+ sTn+ cells, CD19−CD5+sTn+ cells, CD19−CD21+ cells, and a combination thereof.

80. The method according to claim 78, wherein an affinity ligand further comprises a detectable moiety.

81. The method according to claim 78, wherein the clinical sample comprises peripheral blood.

82. The method according to claim 79, wherein the clinical sample comprises peripheral blood.

83. The method according to claim 81, wherein a difference in the amount of mononuclear cell phenotype determined from the clinical sample, as compared to a reference value for the mononuclear cell phenotype, comprises a difference selected from the group consisting of an increase in CD19+sTn+ cells, an increase in CD19+CD21+ cells, an increase in CD19+CD21++ cells, a decrease in CD19+CD21+sTn+ cells, an increase in CD19−CD21+sTn+ cells, an increase in CD19−CD21+ cells, and a combination thereof.

84. The method according to claim 82, wherein a difference in the amount of mononuclear cell phenotype determined from the clinical sample, as compared to a reference value for the mononuclear cell phenotype, comprises a difference selected from the group consisting of an increase in CD19+sTn+ cells, an increase in CD19+CD21+ cells, an increase in CD19+CD21++ cells, a decrease in CD19+CD21+sTn+ cells, an increase in CD19+CD5+sTn+ cells, an increase in CD19−CD5+sTn+ cells, an increase in CD19−CD21+sTn+ cells, an increase in CD19−CD21+ cells, and a combination thereof.

85. The method of claim 83, wherein the difference in the amount of mononuclear cell phenotype determined from the clinical sample, as compared to a reference value for the mononuclear cell phenotype, further comprises a decrease in CD19+ cells.

86. The method of claim 84, wherein the difference in the amount of mononuclear cell phenotype determined from the clinical sample, as compared to a reference value for the mononuclear cell phenotype, further comprises a decrease in CD19+ cells.

87. The method according to claim 78, wherein the clinical sample comprises lymphoid tissue.

88. The method according to claim 87, wherein a difference in the amount of mononuclear cell phenotype determined from the clinical sample, as compared to a reference value for the mononuclear cell phenotype, comprises a difference selected from the group consisting of an increase in CD19+sTn+ cells, an increase in CD19+CD21+ cells, an increase in CD19+CD21++ cells, an increase in CD19−CD21+sTn+ cells, and a combination thereof.

89. The method of claim 88, wherein the difference in the amount of mononuclear cell phenotype determined from the clinical sample, as compared to a reference value for the mononuclear cell phenotype, further comprises a difference selected from the group consisting of an increase in CD19+ cells, an increase in CD19−CD21+ cells, and a combination thereof.

90. A method for determining a state of a pro-tumor immune response in an individual, by assaying a clinical sample comprising a reference sample obtained from the individual, and assaying a clinical sample comprising a test sample obtained from the individual subsequent to obtaining the reference sample, the method comprising:
(a) contacting the clinical samples with a plurality of affinity ligands for detecting a mononuclear cell phenotype,
(i) wherein the plurality of affinity ligands comprise an affinity ligand having binding specificity for CD19, an affinity ligand having binding specificity for sTn, and an affinity ligand having binding specificity for CD21, and
(ii) wherein a mononuclear cell phenotype is selected from the group consisting of CD19+sTn+ cells, CD19+CD21+sTn+ cells, CD19+CD21+ cells, CD19+CD21++ cells, CD19−CD21+sTn+ cells, CD19−CD21+ cells, and a combination thereof;
(b) comparing the amount of mononuclear cell phenotype determined from the test sample to the amount of mononuclear cell phenotype determined from the reference sample; and
(c) wherein a difference in the amount of mononuclear cell phenotype determined from the test sample as compared to the amount of mononuclear cell phenotype determined from the reference sample comprises a prognostic indicator for the state of the pro-tumor immune response at a time at which the test sample was obtained from the individual.

91. The method according to claim 90, wherein the plurality of affinity ligands further comprises an affinity ligand having binding specificity for CD5, and wherein a mononuclear cell phenotype is selected from the group consisting of CD19+sTn+ cells, CD19+CD21+sTn+ cells, CD19+CD21+ cells, CD19+CD21++ cells, CD19−CD21+sTn+ cells, CD19−CD5+ sTn+ cells, CD19−CD5+sTn+ cells, CD19−CD21+ cells, and a combination thereof.

92. The method according to claim 90, wherein an affinity ligand further comprises a detectable moiety.

93. The method according to claim 90, wherein the clinical samples comprise peripheral blood.

94. The method according to claim 91, wherein the clinical samples comprise peripheral blood.

95. The method according to claim 93, wherein a difference in the amount of mononuclear cell phenotype determined from the test sample, as compared to the amount of mononuclear cell phenotype determined from the reference sample, comprises a difference in a mononuclear cell phenotype selected from the group consisting of CD19+sTn+ cells, CD19+CD21+ cells, CD19+CD21++ cells, CD19+CD21+sTn+ cells, CD19−CD21+sTn+ cells, CD19−CD21+ cells, and a combination thereof.

96. The method according to claim 94, wherein a difference in the amount of mononuclear cell phenotype determined from the test sample, as compared to the amount of mononuclear cell phenotype determined from the reference sample, comprises a difference in a mononuclear cell phenotype selected from the group consisting of CD19+sTn+ cells, CD19+CD21+ cells, CD19+CD21++ cells, CD19+CD21+sTn+ cells, CD19+CD5+sTn+ cells, CD19−CD5+ sTn+ cells, CD19−CD21+sTn+ cells, CD19−CD21+ cells, and a combination thereof.

97. The method of claim 95, wherein the difference in the amount of mononuclear cell phenotype determined from the test sample, as compared to as compared to the amount of mononuclear cell phenotype determined from the reference sample, further comprises a mononuclear cell phenotype comprising CD19+ cells.

98. The method of claim 90, wherein the difference in the amount of mononuclear cell phenotype determined from the test sample, as compared to as compared to the amount of mononuclear cell phenotype determined from the reference sample, further comprises a mononuclear cell phenotype comprising CD19+ cells.

99. The method according to claim 90, wherein the clinical samples comprise lymphoid tissue.

100. The method according to claim 99, wherein a difference in the amount of mononuclear cell phenotype determined from the test sample, as compared to the amount of mononuclear cell phenotype determined from the reference sample, comprises a difference in a mononuclear cell phenotype selected from the group consisting of CD19+sTn+ cells, CD19+CD21+ cells, CD19+CD21++ cells, CD19−CD21+sTn+ cells, and a combination thereof.

101. The method of claim 100, wherein the difference in the amount of mononuclear cell phenotype determined from the test sample, as compared to as compared to the amount of mononuclear cell phenotype determined from the reference sample, further comprises a mononuclear cell phenotype selected from the group consisting of CD19+ cells, CD19−CD21+ cells, and a combination thereof.

102. The method of claim 90, wherein the method is used to monitor efficacy of anticancer therapy, wherein the reference sample is obtained from the individual at a time selected from the group consisting of before anticancer therapy is initiated, and during anticancer therapy; and wherein the test sample is obtained from the individual at a time selected from the group consisting of a time subsequent to the obtaining of the reference sample, and after conclusion of anticancer therapy.
